(19) United States
(12) Patent Application Publication
(10) Pub. No.: US 2009/0004296 A1
(43) Pub. Date: Jan. 1, 2009

(54) Antiseptic Compositions and Methods of Using Same

(75) Inventor: Eran Gabbai, Kfar-MaAs (IL)

Correspondence Address: FOLEY AND LARDNER LLP;SUITE 500, 3000 K STREET NW, WASHINGTON, DC, 20007, US

(73) Assignee: Do-Coop Technologies LTD.

(21) Appl. No.: 12/087,431

(22) PCT Filed: Jan. 4, 2007
(86) PCT No.: PCT/IL2007/000015

§ 371 Date: Jul. 3, 2008

Related U.S. Application Data
(63) Continuation of application No. 11/324,586, filed on Jan. 4, 2006.
(60) Provisional application No. 60/755,852, filed on Jan. 4, 2006.
(60) Provisional application No. 60/755,850, filed on Jan. 4, 2006.
(60) Provisional application No. 60/755,851, filed on Jan. 4, 2006.

(51) Int. Cl. A61K 33/18; A61K 47/04; A61P 31/02

(52) U.S. Cl. 424/667; 514/769

(57) ABSTRACT

A novel antiseptic composition comprising an antiseptic in a carrier composition comprising nanostructures and a liquid and methods of use thereof are provided.

### FIELD AND BACKGROUND OF THE INVENTION

The present invention relates to a novel antiseptic composition and methods of using same.

Infections are a significant problem in many fields where sanitary conditions are important, such as in healthcare. Problematic infections may arise from bacterial, fungal, amoebic, protozoan and/or viral organisms. Challenges are encountered both in preventing infection, and in reducing or eliminating the infection once it is established. Infected environments may include surfaces of objects, fluids and fluid conduits and/or biological environments such as of human and animals.

Antiseptic agents kill or inhibit the growth of microorganisms on the external surfaces of the body. Common antiseptics include alcohol, iodine, hydrogen peroxide, and boric acid. There is great variation in the ability of antiseptics to destroy microorganisms and in their effect on living tissue. For example, mercuric chloride is a powerful antiseptic, but it irritates delicate tissue. In contrast, silver nitrate kills fewer germs but can be used on the delicate tissues of the eyes and throat. There is also a great difference in the time required for different antiseptics to work. Iodine, one of the fastest-working antiseptics, kills bacteria within thirty seconds. Other antiseptics have slower, more residual actions.

Antiseptics are used prior to surgical interventions, prior to injections, punctures and prior to inspections of hollow organs when the skin or the mucous membrane has to be disinfected. In addition, antiseptics are also employed for wound treatment (surgical wounds, chronic wounds, burn wounds, bite wounds, cut wounds and traumatogenic wounds) and for the therapy of local superficial skin infections (e.g. in fungal infections). Solutions containing antiseptics may be used for caries prophylaxis in the form of mouthwashes. Irrigation (e.g. bladder and abdominal irrigation) is also affected in the presence of antiseptics. Special application areas include prophylactic, preoperative and therapeutic ophthalmic antisepsis, antisepsis of the oral cavity before maxillary surgical interventions and tooth extractions and in infections in the neck and pharyngeal space.

Alcohol-based antiseptics for use in dermal applications such as surgical scrubs, preoperative skin preparations and antiseptic hand washes are well known and widely used because of their high effectiveness and the speed with which they kill microorganisms, as well as their non cytotoxicity. Alcohol containing formulations, containing 60-95% by volume of ethanol or isopropanol, are often used as surgical scrubs, in preoperative skin preparations, as healthcare personnel hand washes and antiseptic hand washes to disinfect hands, and for localized skin disinfection at the site of an invasive medical procedure. The efficacy of such compositions is short term, due to rapid evaporation of the alcohol, which is the antimicrobially active ingredient. Other limitations resulting from the use of such formulations include skin dryness and difficulty in application due to their low viscosity and watery nature. Their use in applications requiring sustained antimicrobial efficacy (persistence), such as surgical scrubs, is therefore limited by their high vapor pressure (which causes rapid evaporation upon application). Thus, when applied to skin, the rapid decrease in alcohol concentration limits the agent's contact time with microbes, especially bacteria, due to evaporative loss.

Antiseptic mouthwashes have been extensively used for centuries and act to is kill bacteria in the oral cavity that are responsible for plaque, gingivitis and bad breath. Mouthwashes, such as Listerine™ (Pfizer) comprise the active ingredients thymol, methyl salicylate, menthol and eucalyptol, albeit in very minute amounts. Without being bound to theory, it is believed that the efficacy and taste of antiseptic mouthwashes such as Listerine™ is due to the availability or dissolution of these four active ingredients. Dissolution is also important from an aesthetic point of view in that a clear amber-colored mouthwash solution is certainly preferred by consumers to one that is cloudy or turbid or heterogeneous. In the majority of mouthwashes including Listerine™, ethanol is used as the solvent. Since ethanol is present in concentrations between 21 and 26% w/v it contributes to the antiseptic efficacy of the mouthwash.

Alcohol-containing mouthwashes are disadvantageous as they may cause burning or stinging effects in the mouth of the user, and additionally may predispose the mouth and throat to cancers (Weaver et al., J Oral Surg. 1979 April; 37(4):250-3; 3 Zunt et al., Indiana Dent Assoc. 1991 November-December; 70(6):16-9). Furthermore, alcohol-containing mouthwashes may be problematic for some users including those who cannot, or should not use alcohol because of physiological, psychological, social or job related reasons. Alcohol is absorbed sublingually. It has been documented that, although mouthwashes should be expectorated, alcoholics, are likely to be abusers of any substance containing alcohol, including mouthwashes.

It is known that patients undergoing chemotherapy should not ingest even minute amounts of alcohols. Chemotherapy causes the parotid glands to produce an insufficient amount of saliva and a dry mouth. Use of alcohol containing mouthwashes exacerbates this problem. Therefore, rinsing with an alcohol-free mouthwash would aid in the dental care of these patients.

There is thus a widely recognized need for, and it would be highly advantageous to have, antiseptic compositions devoid of the above-limitations.

### SUMMARY OF THE INVENTION

According to one aspect of the present invention there is provided an antiseptic composition comprising at least one antiseptic agent and a carrier composition comprising nanostructures and a liquid.

According to another aspect of the present invention there is provided a method of disinfecting a body surface of an individual comprising providing to an individual in need thereof an antiseptic effective amount of a composition wherein the composition comprises nanostructures and a liquid, thereby disinfecting a body surface of an individual.

According to yet another aspect of the present invention there is provided a method of sterilizing an object comprising contacting the object with a composition comprising nanostructures and a liquid, thereby sterilizing the object.

According to further features in preferred embodiments of the invention described below, the composition further comprises at least one antiseptic agent.

According to still further features in the described preferred embodiments, the nanostructures comprise a core material of a nanometric size enveloped by ordered fluid molecules of the liquid, the core material and the envelope of ordered fluid molecules being in a steady physical state.

According to still further features in the described preferred embodiments the fluid molecules comprise a heterogeneous fluid composition comprising at least two homogeneous fluid compositions and whereas the liquid is identical to at least one of the at least two homogeneous fluid compositions.

According to still further features in the described preferred embodiments the fluid molecules are in a gaseous state.

According to still further features in the described preferred embodiments a concentration of the nanostructures is less than 10²⁰ per liter.

According to still further features in the described preferred embodiments a concentration of the nanostructures is less than 10¹⁵ per liter

According to still further features in the described preferred embodiments the nanostructures are capable of forming clusters.

According to still further features in the described preferred embodiments the nanostructures are capable of maintaining long range interaction thereamongst.

According to still further features in the described preferred embodiments the core material is selected from the group consisting of a ferroelectric material, a ferromagnetic material and a piezoelectric material.

According to still further features in the described preferred embodiments the core material is a crystalline core material.

According to still further features in the described preferred embodiments the liquid is water.

According to still further features in the described preferred embodiments each of the nanostructures is characterized by a specific gravity lower than or equal to a specific gravity of the liquid.

According to still further features in the described preferred embodiments the composition is characterized by an enhanced ultrasonic velocity relative to water.

According to still further features in the described preferred embodiments the composition comprises a buffering capacity greater than a buffering capacity of water.

According to still further features in the described preferred embodiments the nanostructures are formulated from hydroxyapatite.

According to still further features in the described preferred embodiments the antiseptic composition is formulated as a liquid composition.

According to still further features in the described preferred embodiments the liquid composition comprises at least 1% by volume of the carrier composition.

According to still further features in the described preferred embodiments the antiseptic composition is formulated as a solid composition.

According to still further features in the described preferred embodiments the solid composition comprises at least 0.258 gr/100 ml of the carrier composition.

According to still further features in the described preferred embodiments the antiseptic composition is formulated as an oral dosage form.

According to still further features in the described preferred embodiments the oral dosage form is selected from the group consisting of a mouthwash, a strip, a foam, a chewing gum, an oral spray, a lozenge and a capsule.

According to still further features in the described preferred embodiments the antiseptic composition is formulated as a topical or mucosal dosage form.

According to still further features in the described preferred embodiments the topical or mucosal dosage form is selected from the group consisting of a cream, a spray, a wipe, a foam, a soap, an oil, a solution, a lotion, an ointment, a paste and a gel.

According to still further features in the described preferred embodiments the antiseptic composition comprises less than 20% by volume alcohol.

According to still further features in the described preferred embodiments the antiseptic agent is an orally non-toxic antiseptic agent.

According to still further features in the described preferred embodiments the orally non-toxic antiseptic agent is selected from the group consisting of thymol, methyl salicylate, menthol, sodium chloride, hydrogen peroxide, chlorhexidine gluconate, chlorobutanol hemihydrate, phenol, eucalyptol.

According to still further features in the described preferred embodiments the at least one antiseptic agent is selected from the group consisting of a monohydric alcohol, a metal compound, a quaternary ammonium compound, iodine, an iodophor and a phenolic compound.

According to still further features in the described preferred embodiments the monohydric alcohol is selected from the group consisting of ethanol and isopropanol.

According to still further features in the described preferred embodiments the metal compound is selected from the group consisting of silver nitrate and silver sulfadiazine.

According to still further features in the described preferred embodiments the quaternary ammonium compound is selected from the group consisting of diethyl benzyl ammonium chloride, benzalkonium chloride, diethyl dodecyl benzyl ammonium chloride, dimethyl didodecyl ammonium chloride, octadecyl dimethyl benzyl ammonium chloride, trimethyl tetradecyl ammonium chloridem, trimethyl octadecylammonium chloride, trimethyl hexadecyl ammonium chloride, Alkyl dimethyl benzyl ammonium chloride, cetyl pyridinium bromide, cetyl pyridinium chloride, dodecylpyridinium chloride, and benzyl dodecyl bis(B-hydroxyethyl) ammonium chloride.

According to still further features in the described preferred embodiments the phenolic compound is selected from the group consisting of phenol, para-chlorometaxylenol, cresol and hexylresorcinol.

According to still further features in the described preferred embodiments the body surface is a skin, a tooth or a mucous membrane.

According to still further features in the described preferred embodiments the antiseptic agent is a toxic agent.

According to still further features in the described preferred embodiments the toxic agent is selected from the group consisting of formaldehyde, chlorine, mercuric chloride and ethylene oxide.

The present invention successfully addresses the shortcomings of the presently known configurations by providing novel antiseptic compositions and methods of using same.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B are graphs comparing the absorption of an antiseptic composition in the liquid composition of the present invention (FIG. 1A) and the antiseptic composition in reverse osmosis water (FIG. 1B) at increasing wavelengths following a two-hour incubation period.

FIG. 2 shows results of isothermal measurements of absolute ultrasonic velocity in the liquid composition of the present invention as a function of observation time.

FIG. 3 is a photograph of a plastic apparatus comprising four upper channels and one lower channel connected via capillary channels.

FIGS. 4A-B are photographs of plastic apparatus following addition of a dye and diluting agent to the upper channels. FIG. 4A shows that fifteen minutes following placement there is no movement from the upper channels to the lower channel via the capillaries when the diluting agent is water. FIG. 4B shows that fifteen minutes following placement, there is movement from the upper channels to the lower channel via the capillaries when the diluting agent is the liquid composition of the present invention.

FIG. 5 is a graph illustrating Sodium hydroxide titration of various water compositions as measured by absorbence at 557 nm.

FIGS. 6A-C are graphs of an experiment performed in triplicate illustrating Sodium hydroxide titration of water comprising nanostructures and RO water as measured by pH.

FIGS. 7A-C are graphs illustrating Sodium hydroxide titration of water comprising nanostructures and RO water as measured by pH, each graph summarizing 3 triplicate experiments.

FIGS. 8A-C are graphs of an experiment performed in triplicate illustrating Hydrochloric acid titration of water comprising nanostructures and RO water as measured by pH.

FIG. 9 is a graph illustrating Hydrochloric acid titration of water comprising nanostructures and RO water as measured by pH, the graph summarizing 3 triplicate experiments.

FIGS. 10A-C are graphs illustrating Hydrochloric acid (FIG. 10A) and Sodium hydroxide (FIGS. 10B-C) titration of water comprising nanostructures and RO water as measured by absorbence at 557 nm.

FIGS. 11A-B are photographs of cuvettes following Hydrochloric acid titration of RO (FIG. 11A) and water comprising nanostructures (FIG. 11B). Each cuvette illustrated addition of 1 μl of Hydrochloric acid.

FIGS. 12A-C are graphs illustrating Hydrochloric acid titration of RF water (FIG. 12A), RF2 water (FIG. 12B) and RO water (FIG. 12C). The arrows point to the second radiation.

FIG. 13 is a graph illustrating Hydrochloric acid titration of FR2 water as compared to RO water. The experiment was repeated three times. An average value for all three experiments was plotted for RO water.

FIGS. 14A-J are photographs of solutions comprising red powder and Neowater™ following three attempts at dispersion of the powder at various time intervals. FIGS. 14A-E illustrate right test tube C (50% EtOH+Neowater™) and left test tube B (dehydrated Neowater™) from Example 8 part C. FIGS. 14G-J illustrate solutions following overnight crushing of the red powder and titration of 100 μl Neowater™

FIGS. 15A-C are readouts of absorbance of 2 μl from 3 different solutions as measured in a nanodrop. FIG. 15A represents a solution of the red powder following overnight crushing+100 μl Neowater. FIG. 15B represents a solution of the red powder following addition of 100% dehydrated Neowater™ and FIG. 15C represents a solution of the red powder following addition of EtOH+Neowater™ M (50%-50%).

FIG. 16 is a graph of spectrophotometer measurements of vial #1 (CD-Dau+Neowater™), vial #4 (CD-Dau+10% PEG in Neowater™) and vial #5 (CD-Dau+50% Acetone+50% Neowater™).

FIG. 17 is a graph of spectrophotometer measurements of the dissolved material in Neowater™ (blue line) and the dissolved material with a trace of the solvent acetone (pink line).

FIG. 18 is a graph of spectrophotometer measurements of the dissolved material in Neowater™ (blue line) and acetone (pink line). The pale blue and the yellow lines represent different percent of acetone evaporation and the purple line is the solution without acetone.

FIG. 19 is a graph of spectrophotometer measurements of CD-Dau at 200-800 nm. The blue line represents the dissolved material in RO while the pink line represents the dissolved material in Neowater™.

FIG. 20 is a graph of spectrophotometer measurements of t-boc at 200-800 nm. The blue line represents the dissolved material in RO while the pink line represents the dissolved material in Neowater™.

FIGS. 21A-D are graphs of spectrophotometer measurements at 200-800 nm n. FIG. 21A is a graph of AG-14B in the presence and absence of ethanol immediately following ethanol evaporation. FIG. 21B is a graph of AG-14B in the presence and absence of ethanol 24 hours following ethanol evaporation. FIG. 21C is a graph of AG-14A in the presence and absence of ethanol immediately following ethanol evaporation. FIG. 21D is a graph of AG-14A in the presence and absence of ethanol 24 hours following ethanol evaporation.

FIG. 22 is a photograph of suspensions of AG-14A and AG14B 24 hours following evaporation of the ethanol.

FIGS. 23A-G are graphs of spectrophotometer measurements of the peptides dissolved in Neowater™. FIG. 23A is a graph of Peptide X dissolved in Neowater™. FIG. 23B is a graph of X-5FU dissolved in Neowater™. FIG. 23C is a graph of NLS-E dissolved in Neowater™. FIG. 23D is a graph of Palm-PFPSYK (CMFU) dissolved in Neowater™. FIG. 23E is a graph of PFPSYKLRPG-NH₂ dissolved in Neowater™. FIG. 23F is a graph of NLS-p2-LHRH dissolved in Neowater™, and FIG. 23G is a graph of F-LH-RH-palm kGFPSK dissolved in Neowater™.

FIGS. 24A-G are bar graphs illustrating the cytotoxic effects of the peptides dissolved in Neowater™ as measured by a crystal violet assay. FIG. 24A is a graph of the cytotoxic effect of Peptide X dissolved in Neowater™. FIG. 24B is a graph of the cytotoxic effect of X-5FU dissolved in Neowater™. FIG. 24C is a graph of the cytotoxic effect of NLS-E dissolved in Neowater™. FIG. 24D is a graph of the cytotoxic effect of Palm—PFPSYK (CMFU) dissolved in Neowater™. FIG. 24E is a graph of the cytotoxic effect of PFPSYKLRPG-NH₂ dissolved in Neowater™.

FIG. 24F is a graph of the cytotoxic effect of NLS-p2-LHRH dissolved in Neowater™, and FIG. 24G is a graph of the cytotoxic effect of F-LH-RH-palm kGFPSK dissolved in Neowater™.

FIG. 25 is a graph of retinol absorbance in ethanol and Neowater™.

FIG. 26 is a graph of retinol absorbance in ethanol and Neowater™ following filtration.

FIGS. 27A-B are photographs of test tubes, the left containing Neowater™ and substance “X” and the right containing DMSO and substance “X”. FIG. 27A illustrates test tubes that were left to stand for 24 hours and FIG. 27B illustrates test tubes that were left to stand for 48 hours.

FIGS. 28A-C are photographs of test tubes comprising substance “X” with solvents 1 and 2 (FIG. 28A), substance “X” with solvents 3 and 4 (FIG. 28B) and substance “X” with solvents 5 and 6 (FIG. 28C) immediately following the heating and shaking procedure.

FIGS. 29A-C are photographs of test tubes comprising substance “X” with solvents 1 and 2 (FIG. 29A), substance “X” with solvents 3 and 4 (FIG. 29B) and substance “X” with solvents 5 and 6 (FIG. 29C) 60 minutes following the heating and shaking procedure.

FIGS. 30A-C are photographs of test tubes comprising substance “X” with solvents 1 and 2 (FIG. 30A), substance “X” with solvents 3 and 4 (FIG. 30B) and substance “X” with solvents 5 and 6 (FIG. 30C) 120 minutes following the heating and shaking procedure.

FIGS. 31A-C are photographs of test tubes comprising substance “X” with solvents 1 and 2 (FIG. 31A), substance “X” with solvents 3 and 4 (FIG. 31B) and substance “X” with solvents 5 and 6 (FIG. 31C) 24 hours following the heating and shaking procedure.

FIGS. 32A-D are photographs of glass bottles comprising substance “X” in a solvent comprising Neowater™ and a reduced concentration of DMSO, immediately following shaking (FIG. 32A), 30 minutes following shaking (FIG. 32B), 60 minutes following shaking (FIG. 32C) and 120 minutes following shaking (FIG. 32D).

FIG. 33 is a graph illustrating the absorption characteristics of material “X” in RO/Neowater™ 6 hours following vortex, as measured by a spectrophotometer.

FIGS. 34A-B are graphs illustrating the absorption characteristics of SPL2101 in ethanol (FIG. 34A) and SPL5217 in acetone (FIG. 34B), as measured by a spectrophotometer.

FIGS. 35A-B are graphs illustrating the absorption characteristics of SPL2101 in Neowater™ (FIG. 35A) and SPL5217 in Neowater™ (FIG. 35B), as measured by a spectrophotometer.

FIGS. 36A-B are graphs illustrating the absorption characteristics of taxol in Neowater™ (FIG. 36A) and DMSO (FIG. 36B), as measured by a spectrophotometer.

FIG. 37 is a bar graph illustrating the cytotoxic effect of taxol in different solvents on 293T cells. Control RO=medium made up with RO water; Control Neo=medium made up with Neowater™; Control DMSO RO=medium made up with RO water+10 μl DMSO; Control Neo RO=medium made up with RO water+10 μl Neowater™; Taxol DMSO RO=medium made up with RO water+taxol dissolved in DMSO; Taxol DMSO Neo=medium made up with Neowater™+taxol dissolved in DMSO; Taxol NW RO=medium made up with RO water+taxol dissolved in Neowater™; Taxol NW Neo=medium made up with Neowater™+taxol dissolved in Neowater™.

FIGS. 38A-B are photographs of a DNA gel stained with ethidium bromide illustrating the PCR products obtained in the presence and absence of the liquid composition comprising nanostructures following heating according to the protocol described in Example 16 using two different Taq polymerases.

FIG. 39 is a photograph of a DNA gel stained with ethidium bromide illustrating the PCR products obtained in the presence and absence of the liquid composition comprising nanostructures following heating according to the protocol described in Example 17 using two different Taq polymerases.

### DESCRIPTION OF THE PREFERRED EMBODIMENTS

The present invention is of a novel antiseptic composition and methods of using same.

Specifically, the present invention can be used to sterilize a body surface (e.g. the mouth, as a mouthwash) or an object.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Antiseptics may be employed for a myriad of purposes including application prior to surgical interventions, prior to injections, punctures and prior to inspections of hollow organs when the skin or the mucous membrane has to be disinfected. In addition, antiseptics are also employed for wound treatment and for the therapy of local superficial skin infections (e.g. in fungal infections). Solutions containing antiseptics may be used for caries prophylaxis in the form of mouthwashes.

Mouthwashes are useful for killing bacteria in the oral cavity that are responsible for plaque, gingivitis and bad breathe. In the majority of mouthwashes, ethanol is used as the solvent. Alcohol-containing mouthwashes are disadvantageous as they may cause burning or stinging effects in the mouth of the user, and additionally are thought to predispose the mouth to cancer. Furthermore, alcohol-containing mouthwashes may be problematic for some users including those who cannot, or should not use alcohol because of physiological (e.g. patients undergoing chemotherapy), psychological, social or job related reasons. Therefore, it is highly desired to have novel antiseptic compositions that are devoid of the above limitations.

While reducing the present invention to practice, the present inventor has uncovered that compositions which comprise nanostructures (such as described in U.S. Pat. Appl. Nos. 60/545,955 and 10/865,955, and International Patent Application, Publication No. WO2005/079153) can be used for disinfecting a body surface or an object either per se or when used as carriers for antiseptic agents.

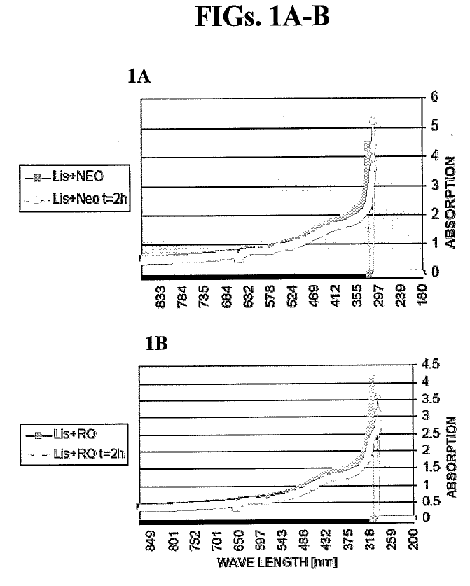

As is illustrated hereinbelow and in the Examples section which follows, the present inventor has shown that the carrier composition of the present invention is effective as a solvent for mouthwash active ingredients (e.g. thymol, methyl salicylate, menthol and eucalyptol). As shown in FIGS. 1 a-b, antiseptic active agents created finer micelles over time, with more dispersion in the carrier composition of the present invention compared with reverse osmosis (RO) water as seen by the higher Optical Density (OD) signal and a curve shift to the right. Since the efficacy and taste of antiseptic mouthwashes, is due to the availability or dissolution of their active ingredients (e.g. thymol, methyl salicylate, menthol and eucalyptol), carrier compositions of the present invention may be an effective solvent for the active ingredients contained in mouthwashes. Furthermore, compositions of the present invention may be alcohol free since no additional alcohol was required for dispersion. The compositions of the present invention may therefore be used as a replacement of alcohol as a solvent.

Thus, according to one aspect of the present invention there is provided an antiseptic composition comprising at least one antiseptic agent and a carrier composition comprising nanostructures and a liquid.

As used herein, the phrase “antiseptic composition” refers to a solid, semi solid or liquid composition which is cytostatic and/or cytotoxic to pathogens such as bacteria, fungi, amoebas, protozoas and/or viruses. Preferably, the antiseptic composition of this aspect of the present invention does not comprise more than 20% alcohol w/v and even more preferably is devoid of alcohol (for the reasons described hereinabove).

As used herein the phrase “carrier composition” refers to a liquid composition which disperses/dissolves the active ingredients of the antiseptic composition e.g., antiseptic agent. Preferably, the carrier composition does not cause significant irritation when applied to a body surface of an organism and does not abrogate—the biological activity and properties of the dissolved antiseptic agent.

The carrier composition may also have antiseptic properties.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, in which case the nanostructure is referred to as a “continuous nanostructure”, or between several hundreds of picometers to several hundreds of nanometers, in which the nanostructure is referred to as a “discontinuous nanostructure”. Thus, the nanostructure of the present embodiments can comprise a nanoparticle, an arrangement of nanoparticles, or any arrangement of one or more nanoparticles and one or more molecules.

The liquid of the above-described composition is preferably an aquatic liquid e.g., water.

According to one preferred embodiment of this aspect of the present invention the nanostructures of the carrier composition comprise a core material of a nanometer size enveloped by ordered fluid molecules, which are in a steady physical state with the core material and with each other. Such a carrier composition is described in U.S. Pat. Appl. Nos. 60/545,955 and 10/865,955 and International Pat. Appl. Publication No. WO2005/079153 to the present inventor, the contents of which are incorporated herein by reference.

Examples of such core materials include, without being limited to, a ferroelectric material, a ferromagnetic material and a piezoelectric material. A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. Preferably, the nanostructures retains the ferroelectric or ferromagnetic properties of the core material, thereby incorporating a particular feature in which macro scale physical properties are brought into a nanoscale environment.

The core material may also have a crystalline structure.

As used herein, the phrase “ordered fluid molecules” refers to an organized arrangement of fluid molecules which are interrelated, e.g., having correlations thereamongst. For example, instantaneous displacement of one fluid molecule can be correlated with instantaneous displacement of one or more other fluid molecules enveloping the core material.

As used herein, the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

Preferably, the ordered fluid molecules of the envelope are identical to the liquid molecules of the carrier composition. The fluid molecules of the envelope may comprise an additional fluid which is not identical to the liquid molecules of the carrier composition and as such the envelope may comprise a heterogeneous fluid composition.

Due to the formation of the envelope of ordered fluid molecules, the nanostructures of the present embodiment preferably have a specific gravity that is lower than or equal to the specific gravity of the liquid.

The fluid molecules may be either in a liquid state or in a gaseous state or a mixture of the two.

A preferred concentration of the nanostrucutures is below 10²⁰ nanostructures per liter and more preferably below 10¹⁵ nanostructures per liter. Preferably a nanostructure in the carrier liquid is capable of clustering with at least one additional nanostructure due to attractive electrostatic forces between them. Preferably, even when the distance between the nanostructures prevents cluster formation (about 0.5-10 μm), the nanostructures are capable of maintaining long-range interactions.

The long-range interaction of the nanostructures has been demonstrated by the present Inventor (see Example 2 in the Examples section that follows). The carrier composition of the present embodiment was subjected to temperature change and the effect of the temperature change on ultrasonic velocity was investigated. As will be appreciated by one of ordinary skill in the art, ultrasonic velocity is related to the interaction between the nanostructures in the composition. As demonstrated in the Examples section that follows, the carrier composition of the present invention is characterized by an enhanced ultrasonic velocity relative to water.

Without being bound to theory, it is believed that the long-range interactions between the nanostructures lends to the unique characteristics of the carrier composition. One such characteristic is that the carrier composition of the present invention is able to dissolve or disperse agents in general and antiseptic agents present in strips in particular to a greater extent than water, as demonstrated in Example 1 and Examples 8-15 of the Example section that follows. Another characteristic is that the carrier composition may also enhance penetration of the antiseptic agent through hydrophobic membranes, as demonstrated in the Example 3 of the Examples section that follows. The carrier composition may also enhance the antiseptic properties of an agent by providing a stabilizing environment. Thus, for example, the present inventors have shown that the carrier composition shields and stabilizes proteins from the effects of heat—Examples 16 and 17; and comprises an enhanced buffering capacity (i.e. greater than the buffering capacity of water)—Examples 4-7.

As used herein, the phrase “buffering capacity” refers to the composition's ability to maintain a stable pH stable as acids or bases are added.

It was found by the inventor of the present invention that the antiseptic properties of the carrier composition are expressed or elevated when the composition contacts specific materials, in particular specific biological materials which are typically present in the upper pharynx, (e.g., eukaryotic fungi, protists, methanogenic Archaea or bacteria). On the other hand, no antiseptic properties were observed without presence of such materials. Thus, the carrier composition of the present embodiments has dormant antiseptic properties, in the sense that specific biological materials serve as “primers” to the antiseptic process.

Production of the nanostructures according to this aspect of the present invention may be carried out using a “top-down” process. The process comprises the following method steps, in which a solid powder (e.g., a mineral, a ceramic powder, a glass powder, a metal powder, or a synthetic polymer) is heated, to a sufficiently high temperature, preferably more than about 700° C.

Examples of solid powders which are contemplated include, but are not limited to, BaTiO₃, WO₃ and Ba₂F₉O₁₂. Unexpectedly, the present inventors have also shown that hydroxyapatite (HA) may also be heated to produce the liquid composition of the present invention. Hydroxyapatite is specifically preferred as it is characterized by intoxocicty and is generally FDA approved for human therapy.

It will be appreciated that many hydroxyapatite powders are available from a variety of manufacturers such as from Sigma, Aldrich and Clarion Pharmaceuticals (e.g. Catalogue No. 1306-06-5).

As shown in Table 2, liquid compositions based on HA, all comprised enhanced buffering capacities as compared to water.

The heated powder is then immersed in a cold liquid, (water), below its density anomaly temperature, e.g., 3° C. or 2° C. Simultaneously, the cold liquid and the powder are irradiated by electromagnetic RF radiation, preferably above 500 MHz, which may be either continuous wave RF radiation or modulated RF radiation.

As mentioned hereinabove, the antiseptic composition of this aspect of the present invention comprises at least one antiseptic agent.

As used herein the phrase “antiseptic agent” refers to an agent which is cytostatic and/or cytotoxic to pathogens such as bacteria, fungi, amoebas, protozoas and/or viruses.

The antiseptic agent of the antiseptic compositions of the present invention is selected according to the intended use of the antiseptic compositions of the present invention.

Preferably, the antiseptic agent is stable over a reasonably long shelf-life (e.g. two years), and preferably it should preferably possess substantivity, i.e. a prolonged contact time between the agent and the microbes on which the agent is to induce its effect.

Thus for example, when the antiseptic composition is used for animate administration, the antiseptic agent is preferably a non-toxic antiseptic agent. For example, when used as a mouthwash, the antiseptic agent of this aspect of the present invention is preferably an orally non-toxic antiseptic agent.

As used herein, the phrase “an orally non-toxic antiseptic agent” refers to an antiseptic agent, which is safe (i.e. does not cause unwanted side-effects) at its recommended dose, and when it is administered as directed. For example, if used in a mouthwash, an orally non-toxic antiseptic agent should be non-toxic when rinsed in the mouth, even if a fraction of the antiseptic agent is swallowed whilst rinsing. Oral antiseptic compositions of the present invention can be used for the treatment and/or prevention of oral diseases such as dental caries, gingivitis, dental infection, abscess and periodontal diseases.

Examples of orally non-toxic antiseptic agents include, but are not limited to thymol, methyl salicylate, menthol, sodium chloride, hydrogen peroxide, chlorohexidine gluconate, chlorobutanol hemihydrate, phenol and eucalyptol.

Other antiseptic agents which may be used by the present invention include, but are not limited to, a monohydric alcohol, a metal compound, a quaternary ammonium compound, iodine, an iodophor or a phenolic compound.

Examples of monohydric alcohols which may be used according to this aspect of the present invention include, but are not limited to ethanol and isopropanol.

Examples of metal compounds which may be used according to this aspect of the present invention include, but are not limited to silver nitrate and silver sulfadiazine.

Examples of quaternary ammonium compound which may be used according to this aspect of the present invention include, but are not limited to diethyl benzyl ammonium chloride, benzalkonium chloride, diethyl dodecyl benzyl ammonium chloride, dimethyl didodecyl ammonium chloride, octadecyl dimethyl benzyl ammonium chloride, trimethyl tetradecyl ammonium chloridem, trimethyl octadecylammonium chloride, trimethyl hexadecyl ammonium chloride, Alkyl dimethyl benzyl ammonium chloride, cetyl pyridinium bromide, cetyl pyridinium chloride, dodecylpyridinium chloride, and benzyl dodecyl bis(B-hydroxyethyl) ammonium chloride.

Examples of phenolic compounds which may be used according to this aspect of the present invention include, but are not limited to phenol, para-chlorometaxylenol, cresol and hexylresorcinol.

The antiseptic composition may also comprise other agents which may be beneficial for a subject. For example, an antibiotic or, in the case of a mouth rinse, the composition may also comprise other agents useful for dental care such as zinc chloride and fluoride derivatives.

As mentioned hereinabove, compositions of the present invention (i.e., carrier composition and/or antiseptic composition) described above are characterized by antiseptic properties and as such can be used for disinfecting or sterilizing objects and body surfaces.

The terms “sterilizing” and “disinfecting” may be used interchangeably and refer to killing, preventing or retarding the growth of pathogens such as bacteria, fungi, amoebas, protozoas and/or viruses.

Examples of objects which can be sterilized using the compositions of the present invention include, but are not limited to a catheter (e.g. vascular catheter, urinary catheter, peritoneal catheter, epidural catheter and central nervous system catheter) a tube (e.g. nephrostomy tube and endrotracheal tube), a stent, an orthopedic device, a prosthetic valve, and a medical implant. Other examples include inorganic surfaces such as floors, table-tops, counter-tops, hospital equipment, wheel chairs, gauze and cotton.

Such objects are contacted with the compositions of the present invention for a period of time (e.g. one minute at room temperature). However, the compositions of the present invention should retain their antiseptic properties at higher temperatures (e.g. 50° C.) so that the objects may be heated in the presence of the antiseptic composition if required.

In order to improve sterilizing efficiency, other agents such as antiseptic agents or cleaning agents (such as a polish, a detergent or an abrasive) can be used. When the antiseptic composition is for inanimate use, the antiseptic agent may be a toxic agent or a non-toxic agent. Examples of toxic antiseptic agents include, but are not limited to formaldehyde, chlorine, mercuric chloride and ethylene oxide. Examples of non-toxic agents are detailed hereinabove.

Alternatively compositions of the present invention can be used for disinfecting a body surface of an individual. This can be effected by providing to the body surface of the individual in need thereof an amount of a composition of the present invention.

In order to improve the disinfection, the method further comprises providing other agents such as antiseptic agents, or other therapeutic agents as detailed hereinabove.

As used herein, the phrase “body surface” refers to a skin, a tooth or a mucous membrane (e.g. the mucous membrane lining the mouth). Preferably, the composition of the present invention does not traverse these body surfaces and enter the circulation.

As used herein, the term “individual” refers to a human or animal subject (i.e., dead or living individuals).

The antiseptic composition of the present invention may also comprise other physiologically acceptable carriers. Additionally, the carrier composition of the present invention may also comprise an excipient or an auxiliary.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Preferably, the antiseptic composition of the present invention is applied locally, e.g. placed on the skin, rinsed in the mouth or gargled in the throat.

Antiseptic compositions of the present invention may be manufactured by processes well known in the art, e.g., by means of conventional mixing, dissolving, granulating, dragee-making, levigating, emulsifying, encapsulating, entrapping or lyophilizing processes. Manufacturing of the nanostructures and liquid is described hereinabove.

Antiseptic compositions for use in accordance with the present invention thus may be formulated in conventional manner. Proper formulation is dependent upon the intended use.

For example, the antiseptic composition of the present invention may be formulated for disinfecting the oral cavity and as such may be formulated as any oral dosage form as long as it is not deliberately swallowed. Examples of oral dosage forms include but are not limited to a mouthwash, a strip, a foam, a chewing gum, an oral spray, a capsule and a lozenge.

The antiseptic composition of the present invention may also be formulated as a topical or mucosal dosage form. Examples of topical or mucosal dosage forms include a cream, a spray, a wipe, a foam, a soap, an oil, a solution, a lotion, an ointment, a paste and a gel.

The antiseptic composition may be formulated as a liquid comprising at least 1% by volume of the carrier composition. Alternatively, the antiseptic composition may be formulated as a solid or semi-solid comprising at most 0.258 gr/100 ml of the carrier composition.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in a mixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The active ingredients for use according to the present invention may be conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Pharmaceutical compositions suitable for use in context of the present invention include compositions wherein the active ingredients are contained in an amount effective to achieve the intended purpose. More specifically, a therapeutically effective amount means an amount of active ingredients (antiseptic agent) effective to disinfect.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of local administrations, with course of treatment lasting from several days to several weeks or until cure is effected or diminution of the disease state is achieved.

The amount of a composition to be locally administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Dispersion of Antiseptic Active Agents in Liquid and Nanostructures

Strips comprising an antiseptic composition (comprising thymol, methyl salicylate, menthol and eucalyptol) were dissolved in both liquid and nanostructures and reverse osmosis water in order to compare their solvent properties.

Materials and Experimental Methods

Materials: Neowater™ (Do-Coop technologies, Israel), reverse osmosis (RO) water, Listerine™ (Pocket Pak) strips (Pfizer Consumer Healthcare, New Jersey).

Method: A strip comprising an antiseptic composition was removed from the package and cut in half. Each half was placed in a vial with 5 ml of either liquid and nanostructures or RO water. Both vials were shaken well for a few seconds and left to stand for a few minutes. The bottles were visually inspected to ensure the strip halves were fully dissolved. OD was measured at t=0 and t=2 hours using a USB 2000 Spectrophotometer (scan 180-850 nm).

Results

Following a two-hour incubation, the antiseptic composition present in the strip created finer micelles over time, with more dispersion in liquid and nanostructures compared with RO water as seen by the higher OD signal and a curve shift to the right (FIGS. 1A-B). Additionally, unlike the RO water, no phase separation was apparent with the liquid and nanostructures.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

### Example 2

### Ultrasonic Tests

The composition of the present invention has been subjected to a series of ultrasonic tests in an ultrasonic resonator.

Methods

Measurements of ultrasonic velocities in the carrier composition of the present invention (referred to in the present Example as Neowater™) and double distilled (dist.) water were performed using a ResoScan® research system (TF Instruments, Heidelberg, Germany).

Calibration

Both cells of the ResoScan® research system were filled with standard water (demin. Water Roth. Art. 3175.2 Charge: 03569036) supplemented with 0.005% Tween 20 and measured during an isothermal measurement at 20° C. The difference in ultrasonic velocity between both cells was used as the zero value in the isothermal measurements as further detailed hereinbelow.

Isothermal Measurements

Cell 1 of the ResoScan® research system was used as reference and was filled with dist. Water (Roth Art. 34781 lot#48362077). Cell 2 was filled with the carrier composition of the present invention. Absolute Ultrasonic velocities were measured at 20° C. In order to allow comparison of the experimental values, the ultrasonic velocities were corrected to 20.000° C.

Results

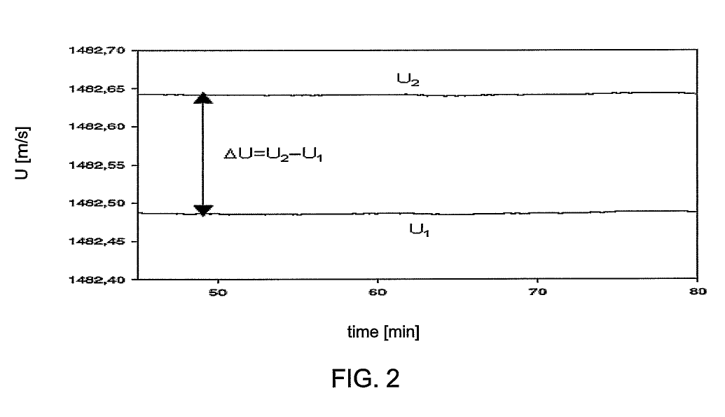

FIG. 2 shows the absolute ultrasonic velocity U as a function of observation time, as measured at 20.051° C. for the carrier composition of the present invention (U₂) and the dist. water (U₁). Both samples displayed stable isothermal velocities in the time window of observation (35 min).

Table 1 below summarizes the measured ultrasonic velocities U₁, U₂ and their correction to 20° C. The correction was calculated using a temperature-velocity correlation of 3 m/s per degree centigrade for the dist. Water.

TABLE 1

| Sample | Temp | U |
|---|---|---|
| dist. water | 20.051° C. | 1482.4851 |
| Neowater ™ | | 1482.6419 |
| dist. water | 20° C. | 1482.6381 |
| Neowater ™ | | 1482.7949 |

As shown in FIG. 2 and Table 1, differences between dist. water and the carrier composition of the present invention were observed by isothermal measurements. The difference ΔU=U₂−U₁ was 15.68 cm/s at a temperature of 20.051° C. and 13.61 cm/s at a temperature of 20° C. The value of ΔU is significantly higher than any noise signal of the ResoScan® system. The results were reproduced on a second ResoScan® research system.

### Example 3

### Hydrophobic Properties of Liquid and Nanostructures

The composition of the present invention was subjected to a series of tests in order to determine if it comprised hydrophobic properties.

Materials and Experimental Methods

Materials: Neowater™ (Do-Coop technologies, Israel); coloring agent Phenol Bromide Blue (Sigma-Aldrich).

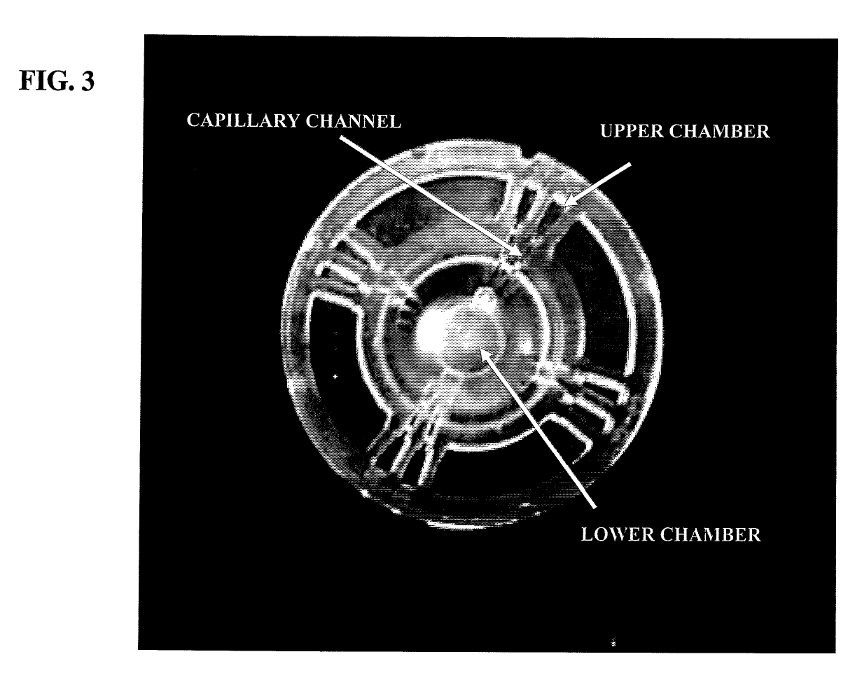

Plastic apparatus: An apparatus was constructed comprising an upper and lower chamber made from a hydrophobic plastic resin (proprietary resin, manufactured by MicroWebFab, Germany). The upper and lower chambers were moulded such that very narrow channels which act as hydrophobic capillary channels interconnect the four upper chambers with the single lower chamber. These hydrophobic capillary channels simulate a typical membrane or other biological barriers (FIG. 3).

Method: The color mix was diluted with the liquid composition of the present invention or with water at a 1:1 dilution. A ten microlitre drop of the liquid composition of the present invention+color composition was placed in the four upper chambers of a first plastic apparatus, whilst in parallel a five hundred microlitre drop of the liquid composition of the present invention was placed in the lower chamber directly above the upper chambers. Similarly a ten microlitre drop of water+color composition was placed in the four upper chambers, of a second plastic apparatus whilst in parallel a five hundred microlitre drop of water was placed in the lower chamber directly above the upper chambers. The location of the dye in each plastic apparatus was analyzed fifteen minutes following placement of the drops.

Results

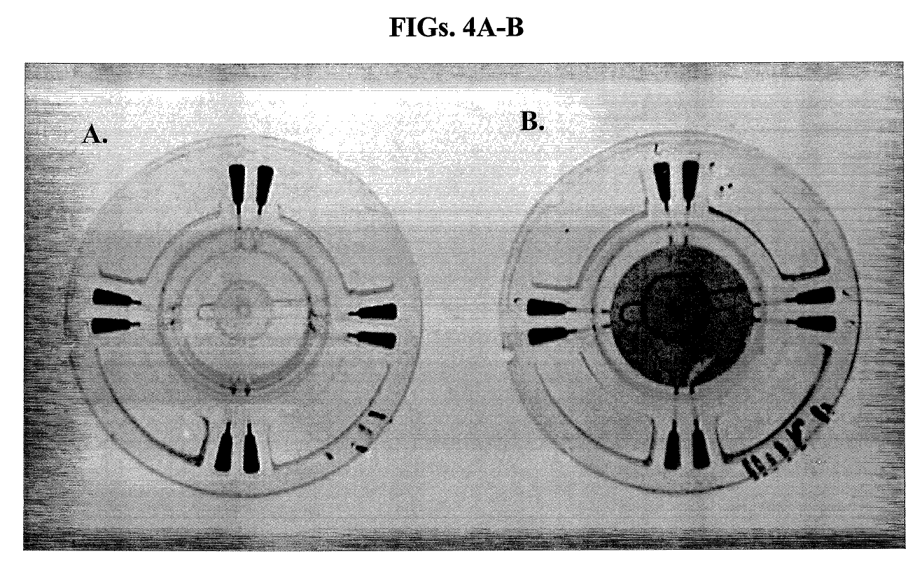

The lower chamber of the plastic apparatus comprising the Water and color mix is clear (FIG. 4A), while the lower chamber of the plastic apparatus comprising the liquid composition of the present invention and color mix, exhibits a light blue color (FIG. 4B).

Conclusion

The liquid composition of the present invention comprises hydrophobic properties as it is able to flow through a hydrophobic capillary.

### Example 4

### Buffering Capacity of the Composition Comprising Nanostructures

The effect of the composition comprising nanostructures on buffering capacity was examined.

Materials and Methods

Phenol red solution (20 mg/25 ml) was prepared. 290 μl was added to 13 ml RO water or various batches of water comprising nanostructures (Neowater™—Do-Coop technologies, Israel). It was noted that each water had a different starting pH, but all of them were acidic, due to their yellow or light orange color after phenol red solution was added. 2.5 ml of each water+phenol red solution were added to a cuvette. Increasing volumes of Sodium hydroxide were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Results

Table 2 summarizes the absorbance at 557 nm of each water solution following sodium hydroxide titration.

TABLE 2

| | | | | | | | μl of 0.02 M |
|---|---|---|---|---|---|---|---|
| NW 1 | NW 2 | NW 3 | NW 4 | NW 5 | | | sodium hydroxide |
| HAP | AB 1-2-3 | HA 18 | Alexander | HA-99-X | NW 6 | RO | added |
| 0.026 | 0.033 | 0.028 | 0.093 | 0.011 | 0.118 | 0.011 | 0 |
| 0.132 | 0.17 | 0.14 | 0.284 | 0.095 | 0.318 | 0.022 | 4 |
| 0.192 | 0.308 | 0.185 | 0.375 | 0.158 | 0.571 | 0.091 | 6 |
| 0.367 | 0.391 | 0.34 | 0.627 | 0.408 | 0.811 | 0.375 | 8 |
| 0.621 | 0.661 | 0.635 | 1.036 | 0.945 | 1.373 | 0.851 | 10 |
| 1.074 | 1.321 | 1.076 | 1.433 | 1.584 | 1.659 | 1.491 | 12 |

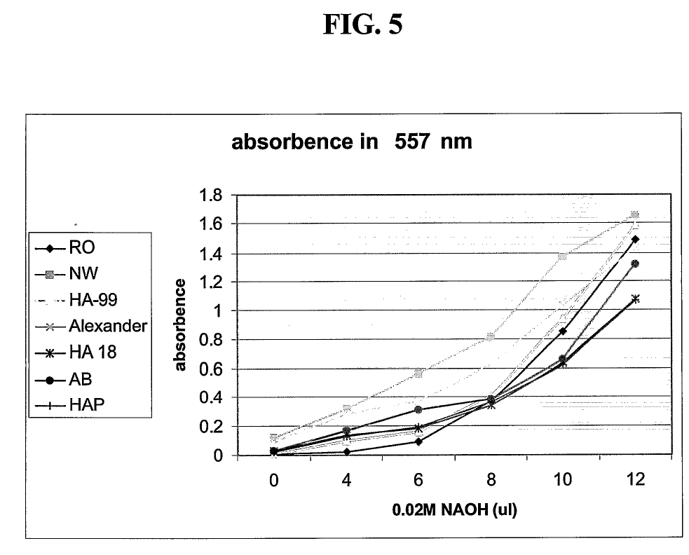

As illustrated in FIG. 5 and Table 2, RO water shows a greater change in pH when adding Sodium hydroxide. It has a slight buffering effect, but when absorbance reaches 0.09 A, the buffering effect “breaks”, and pH change is greater following addition of more Sodium hydroxide. HA-99 water is similar to RO. NW (#150905-106) (Neowater™), AB water Alexander (AB 1-22-1 HA Alexander) has some buffering effect. HAP and HA-18 shows even greater buffering effect than Neowater™.

In summary, from this experiment, all new water types comprising nanostructures tested (HAP, AB 1-2-3, HA-18, Alexander) shows similar characters to Neowater™, except HA-99-X.

### Example 5

### Buffering Capacity of the Liquid Composition Comprising Nanostructures

The effect of the liquid composition comprising nanostructures on buffering capacity was examined.

Materials and Methods

Sodium hydroxide and Hydrochloric acid were added to either 50 ml of RO water or water comprising nanostructures (Neowater™—Do-Coop technologies, Israel) and the pH was measured. The experiment was performed in triplicate. In all, 3 experiments were performed.

Sodium hydroxide titration: −1 μl to 15 μl of 1M Sodium hydroxide was added.

Hydrochloric acid titration: −1 μl to 15 μl of 1M Hydrochloric acid was added.

Results

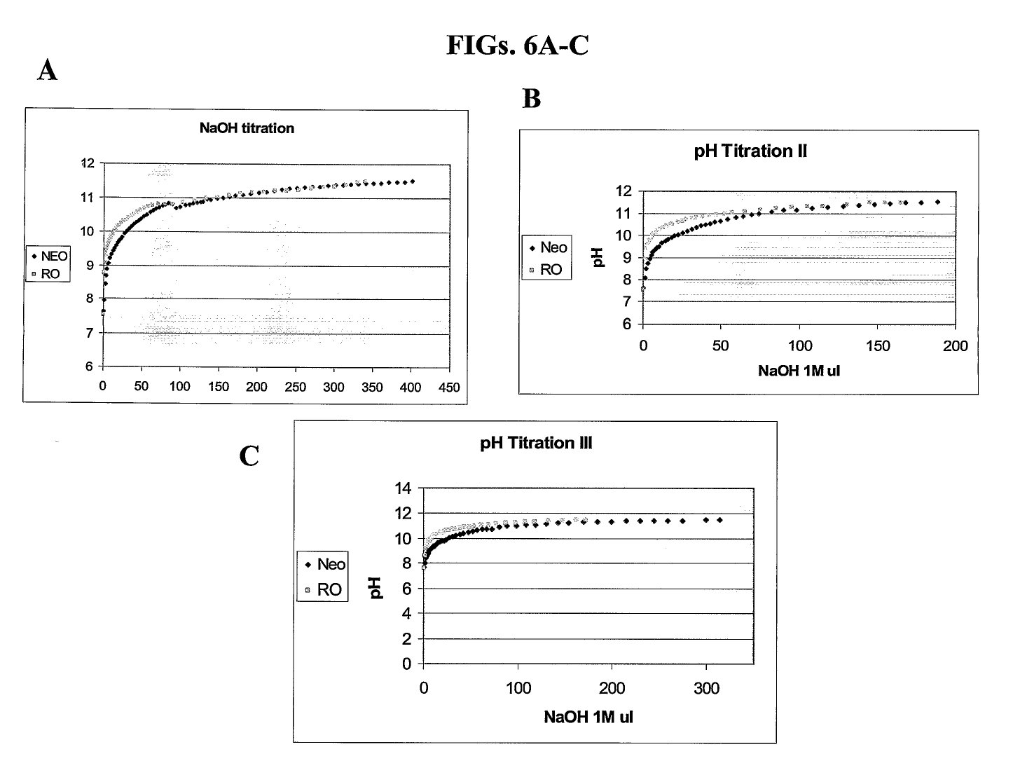

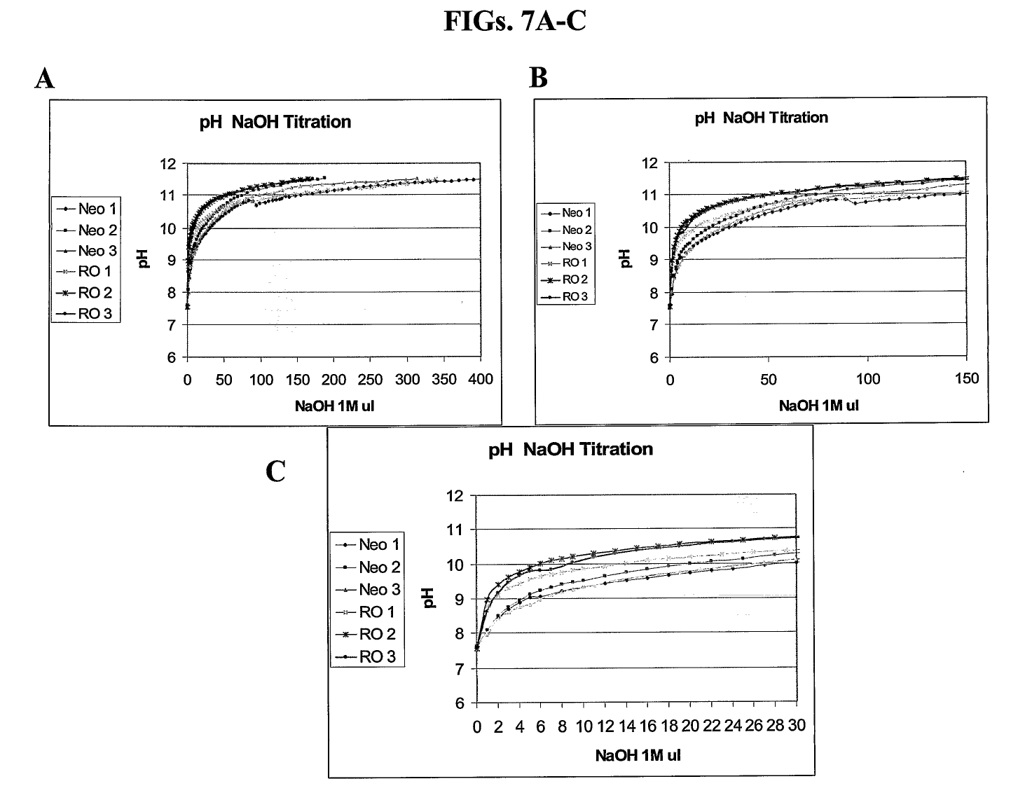

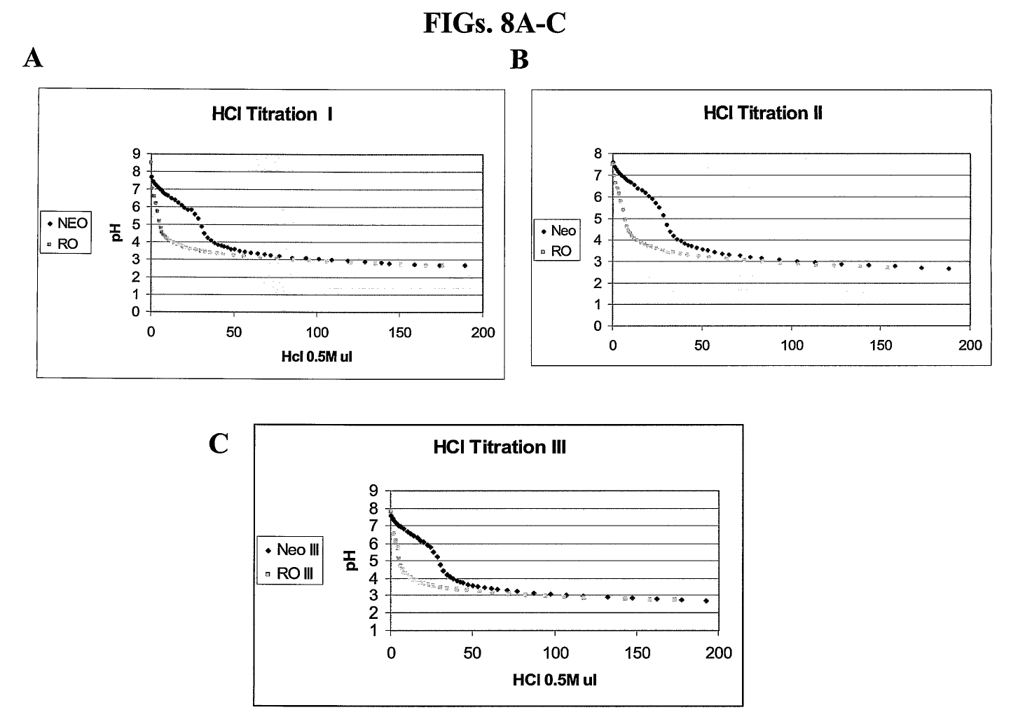

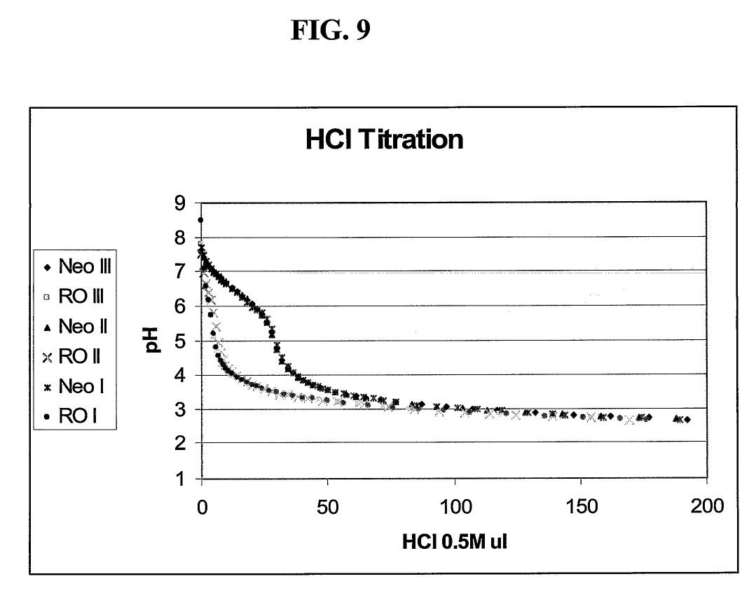

The results for the Sodium hydroxide titration are illustrated in FIGS. 6A-C and 7A-C. The results for the Hydrochloric acid titration are illustrated in FIGS. 8A-C and FIG. 9.

The water comprising nanostructures has buffering capacities since it requires greater amounts of Sodium hydroxide in order to reach the same pH level that is needed for RO water. This characterization is more significant in the pH range of—7.6-10.5. In addition, the water comprising nanostructures requires greater amounts of Hydrochloric acid in order to reach the same pH level that is needed for RO water. This effect is higher in the acidic pH range, than the alkali range. For example: when adding 10 μl Sodium hydroxide 1M (in a total sum) the pH of RO increased from 7.56 to 10.3. The pH of the water comprising nanostructures increased from 7.62 to 9.33. When adding 10 μl Hydrochloric acid 0.5M (in a total sum) the pH of RO decreased from 7.52 to 4.31. The pH of water comprising nanostructures decreased from 7.71 to 6.65. This characterization is more significant in the pH range of −7.7-3.

### Example 6

### Buffering Capacity of the Liquid Composition Comprising Nanostructures

The effect of the liquid composition comprising nanostructures on buffering capacity was examined.

Materials and Methods

Phenol red solution (20 mg/25 ml) was prepared. 1 ml was added to 45 ml RO water or water comprising nanostructures (Neowater™—Do-Coop technologies, Israel). pH was measured and titrated if required. 3 ml of each water+phenol red solution were added to a cuvette. Increasing volumes of Sodium hydroxide or Hydrochloric acid were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Hydrochloric Acid Titration:

RO: 45 ml pH 5.8

1 ml phenol red and 5 μl Sodium hydroxide 1M was added, new pH=7.85 Neowater™ (# 150905-106): 45 ml pH 6.3

1 ml phenol red and 4 μl Sodium hydroxide 1M was added, new pH=7.19 Sodium hydroxide titration:

### I. RO: 45 ml pH 5.78

1 ml phenol red, 6 μl Hydrochloric acid 0.25M and 4 μl Sodium hydroxide 0.5M was added, new pH=4.43 Neowater™ (# 150604-109): 45 ml pH 8.8

1 ml phenol red and 45 μl Hydrochloric acid 0.25M was added, new pH=4.43

### II. RO: 45 ml pH 5.78

1 ml phenol red and 5 μl Sodium hydroxide 0.5M was added, new pH=6.46

### Neowater™ (# 120104-107): 45 ml pH 8.68

1 ml phenol red and 5 μl Hydrochloric acid 0.5M was added, new pH=6.91

Results

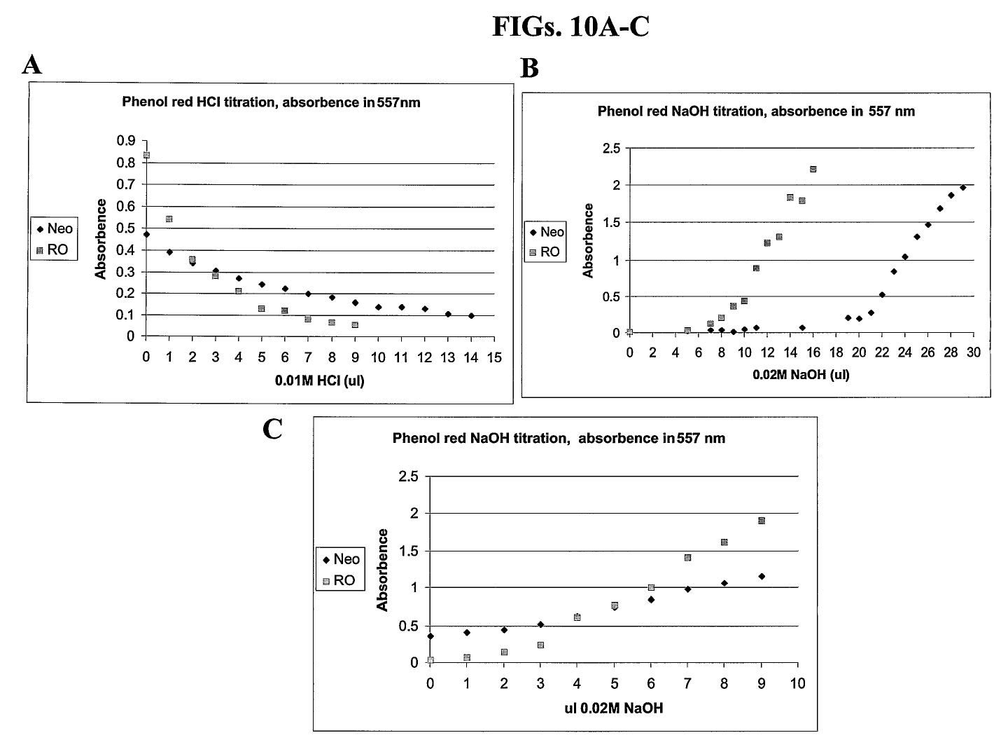

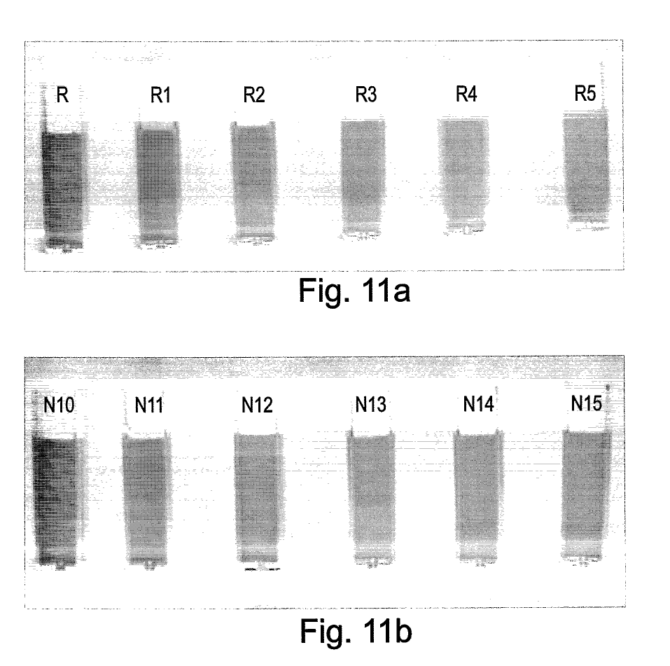

As illustrated in FIGS. 10A-C and 1A-B, the buffering capacity of water comprising nanostructures was higher than the buffering capacity of RO water.

### Example 7

### Buffering Capacity of RF Water

The effect of the RF water on buffering capacity was examined.

Materials and Methods

A few μl drops of Sodium hydroxide 1M were added to raise the pH of 150 ml of RO water (pH=5.8). 50 ml of this water was aliquoted into three bottles.

Three treatments were done:

Bottle 1: no treatment (RO water)

Bottle 2: RO water radiated for 30 minutes with 30 W. The bottle was left to stand on a bench for 10 minutes, before starting the titration (RF water).

Bottle 3: RF water subjected to a second radiation when pH reached 5. After the radiation, the bottle was left to stand on a bench for 10 minutes, before continuing the titration.

Titration was performed by the addition of 1 μl 0.5M Hydrochloric acid to 50 ml water. The titration was finished when the pH value reached below 4.2.

The experiment was performed in triplicates.

Results

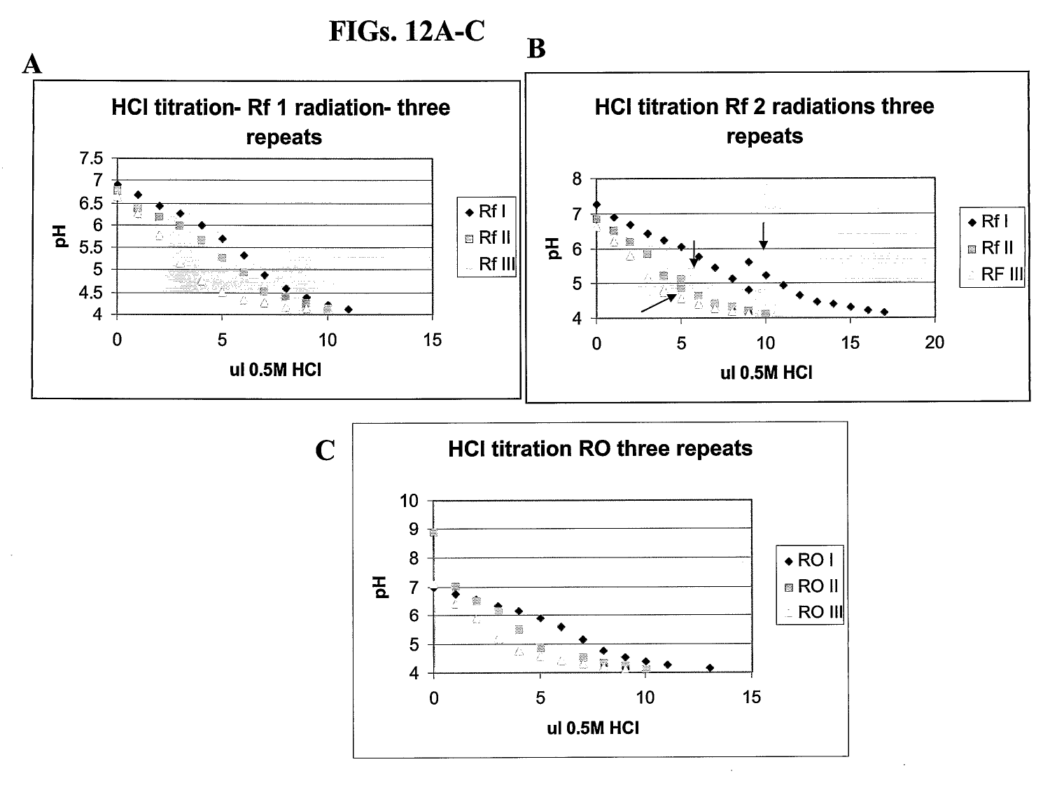

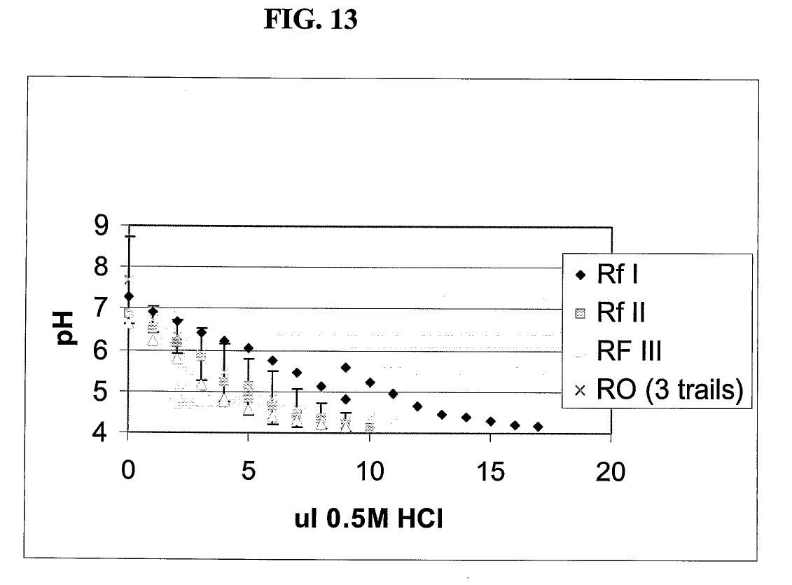

As can be seen from FIGS. 12A-C and FIG. 13, RF water and RF2 water comprise buffering properties similar to those of the carrier composition comprising nanostructures.

### Example 8

### Solvent Capability of the Liquid Composition Comprising Nanostructures

The following experiments were performed in order to ascertain whether the liquid composition comprising nanostructures was capable of dissolving two materials both of which are known not to dissolve in water at a concentration of 1 mg/ml.

A. Dissolving in Ethanol/(Neowater™—Do-Coop Technologies, Israel) Based Solutions

Materials and Methods

Five attempts were made at dissolving the powders in various compositions. The compositions were as follows:

A. 10 mg powder (red/white)+990 μl Neowater™.
B. 10 mg powder (red/white)+990 μl Neowater™ (dehydrated for 90 min).
C. 10 mg powder (red/white)+495 μl Neowater™+495 μl EtOH (50%-50%).
D. 10 mg powder (red/white)+900 μl Neowater™+90 μl EtOH (90%-10%).
E. 10 mg powder (red/white)+820 μl Neowater™+170 μl EtOH (80%-20%).

The tubes were vortexed and heated to 60° C. for 1 hour.

Results
1. The white powder did not dissolve, in all five test tubes.
2. The red powder did dissolve however; it did sediment after a while.
   It appeared as if test tube C dissolved the powder better because the color changed to slightly yellow.

B. Dissolving in Ethanol/(Neowater™—Do-Coop Technologies, Israel) Based Solutions Following Crushing

Materials and Methods

Following crushing, the red powder was dissolved in 4 compositions:

A. ½ mg red powder+49.5 μl RO.
B. ½ mg red powder+49.5 μl Neowater™.
C. ½ mg red powder+9.9 μl EtOH→39.65 μl Neowater™ (20%-80%).
D. ½ mg red powder+24.75 μl EtOH→24.75 μl Neowater™ (50%-50%).
Total reaction volume: 50 μl.

The tubes were vortexed and heated to 60° C. for 1 hour.

Results

Following crushing only 20% of ethanol was required in combination with the Neowater™ to dissolve the red powder.

C. Dissolving in Ethanol/(Neowater™—Do-Coop Technologies, Israel) Solutions Following Extensive Crushing

Materials and Methods

Two crushing protocols were performed, the first on the powder alone (vial 1) and the second on the powder dispersed in 100 μl Neowater™ (1%) (vial 2).

The two compositions were placed in two vials on a stirrer to crush the material overnight:

15 hours later, 100 μl of Neowater™ was added to 1 mg of the red powder (vial no. 1) by titration of 10 μl every few minutes.

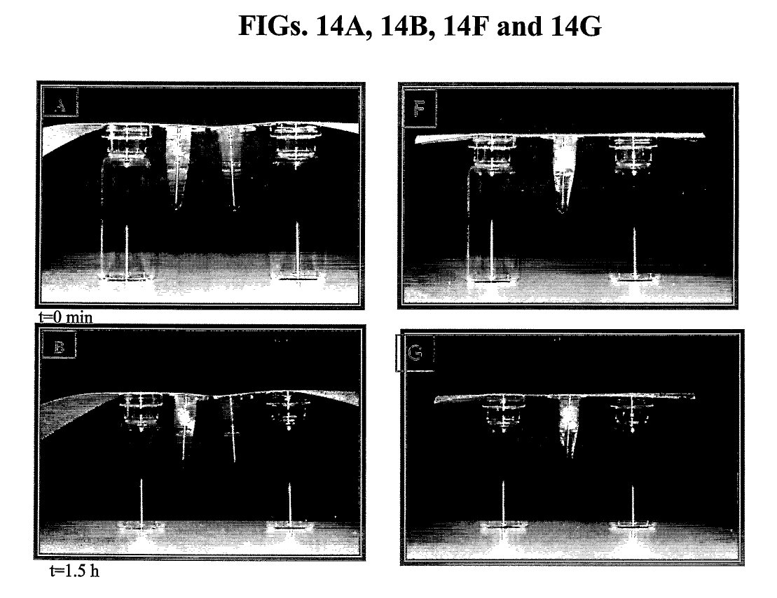

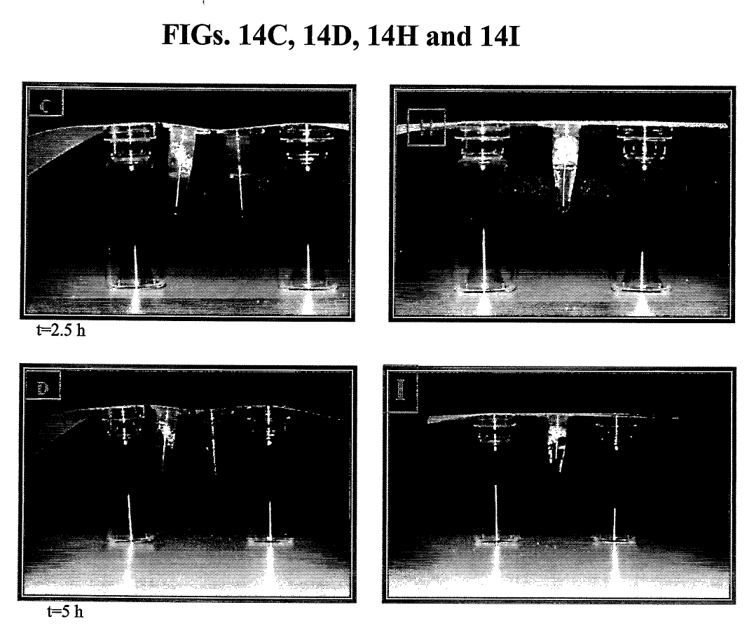

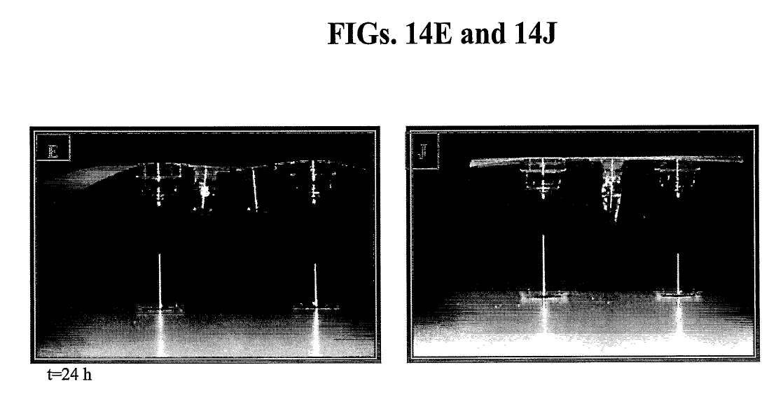

Changes were monitored by taking photographs of the test tubes between 0-24 hours (FIGS. 14F-J).

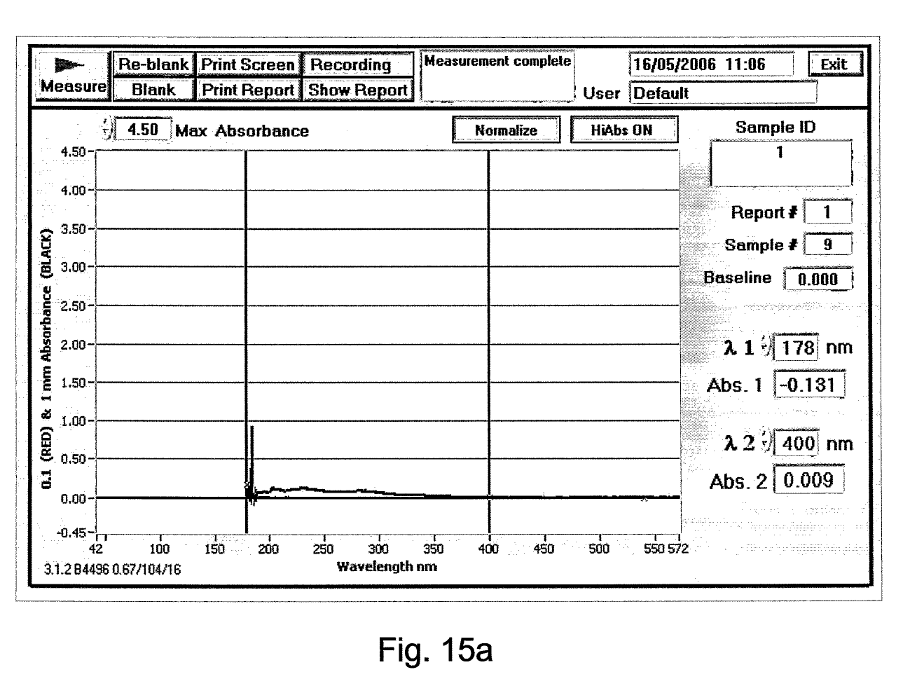

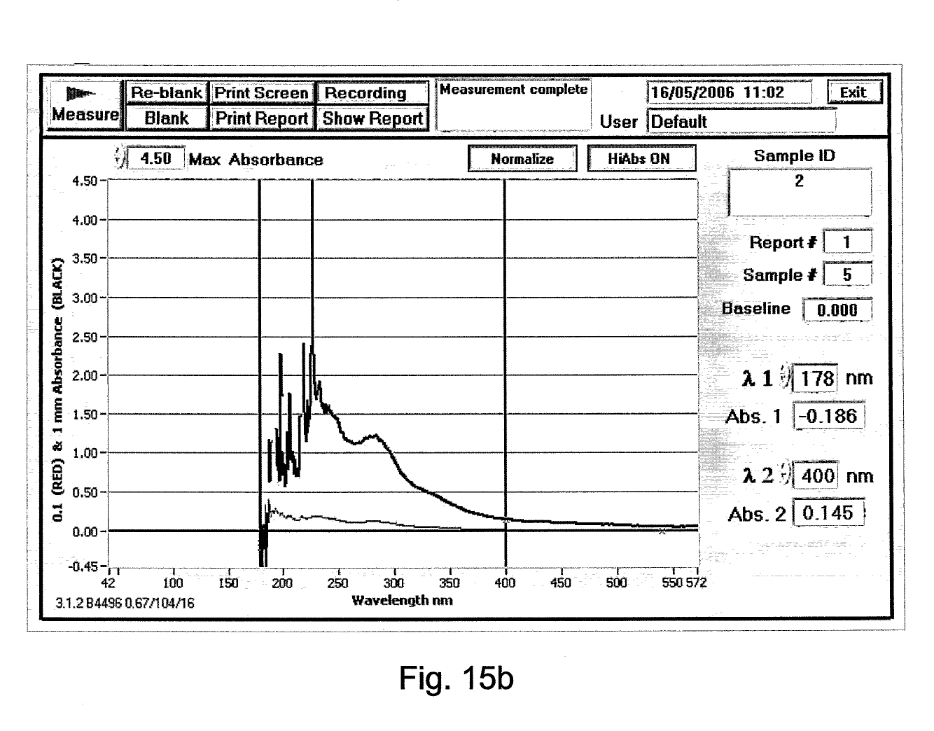

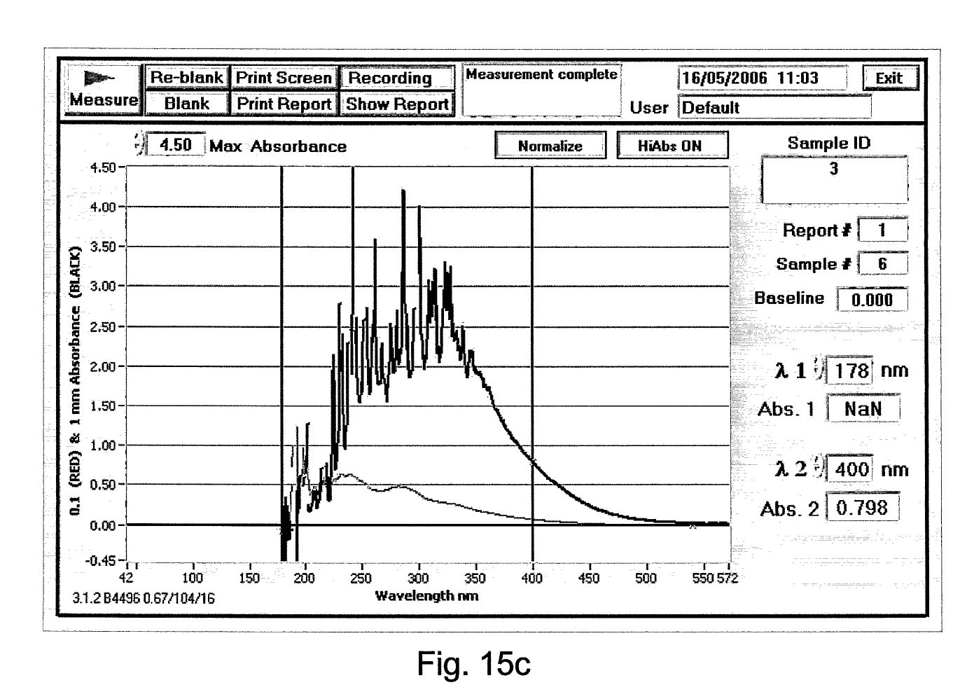

As a comparison, two tubes were observed one of which comprised the red powder dispersed in 990 μl Neowater™ (dehydrated for 90 min)-1% solution, the other dispersed in a solution comprising 50% ethanol/50% Neowater™)-1% solution. The tubes were heated at 60° C. for 1 hour. The tubes are illustrated in FIGS. 14A-E. Following the 24 hour period, 2 μl from each solution was taken and its absorbance was measured in a nanodrop (FIGS. 15A-C)

Results

FIGS. 14A-J illustrate that following extensive crushing, it is possible to dissolve the red material, as the material remains stable for 24 hours and does not sink. FIGS. 14A-E however, show the material changing color as time proceeds (not stable).

Vial 1 almost didn't absorb (FIG. 15A); solution B absorbance peak was between 220-270 nm (FIG. 15B) with a shift to the left (220 nm) and Solution C absorbance peak was between 250-330 nm (FIG. 15C).

Conclusions

Crushing the red material caused the material to disperse in Neowater™. The dispersion remained over 24 hours. Maintenance of the material in glass vials kept the solution stable 72 h later, both in 100% dehydrated Neowater™ and in EtOH-Neowater™ (50%-50%).

### Example 9

### Capability of the Liquid Composition Comprising Nanostructures to Dissolve Daidzein, Daunrubicine and T-Boc Derivative

The following experiments were performed in order to ascertain whether the liquid composition comprising nanostructures was capable of dissolving three materials—Daidzein—daunomycin conjugate (CD-Dau); Daunrubicine (Cerubidine hydrochloride); t-boc derivative of daidzein (tboc-Daid), all of which are known not to dissolve in water.

Materials and Methods

A. Solubilizing CD-Dau—Part 1:

Required concentration: 3 mg/ml Neowater.

Properties: The material dissolves in DMSO, acetone, acetonitrile.
Properties: The material dissolves in EtOH.
5 different glass vials were prepared:
1. 5 mg CD-Dau+1.2 ml Neowater™.
2. 1.8 mg CD-Dau+600 μl acetone.
3. 1.8 mg CD-Dau+150 μl acetone+450 μl Neowater™ (25% acetone).
4. 1.8 mg CD-Dau+600 μl 10% *PEG (Polyethylene Glycol).
5. 1.8 mg CD-Dau+600 μl acetone+600 μl Neowater™.
The samples were vortexed and spectrophotometer measurements were performed on vials #1, 4 and 5

The vials were left opened in order to evaporate the acetone (vials #2, 3, and 5).

Results

Vial #1 (100% Neowater): CD-Dau sedimented after a few hours.

Vial #2 (100% acetone): CD-Dau was suspended inside the acetone, although 48 hours later the material sedimented partially because the acetone dissolved the material.

Vial #3 (25% acetone): CD-Dau didn't dissolve very well and the material floated inside the solution (the solution appeared cloudy).

Vial #4 (10% PEG+Neowater): CD-Dau dissolved better than the CD-Dau in vial #1, however it didn't dissolve as well as with a mixture with 100% acetone.

Vial #5: CD-Dau was suspended first inside the acetone and after it dissolved completely Neowater™ was added in order to exchange the acetone. At first acetone dissolved the material in spite of Neowater™'s presence. However, as the acetone evaporated the material partially sediment to the bottom of the vial. (The material however remained suspended.

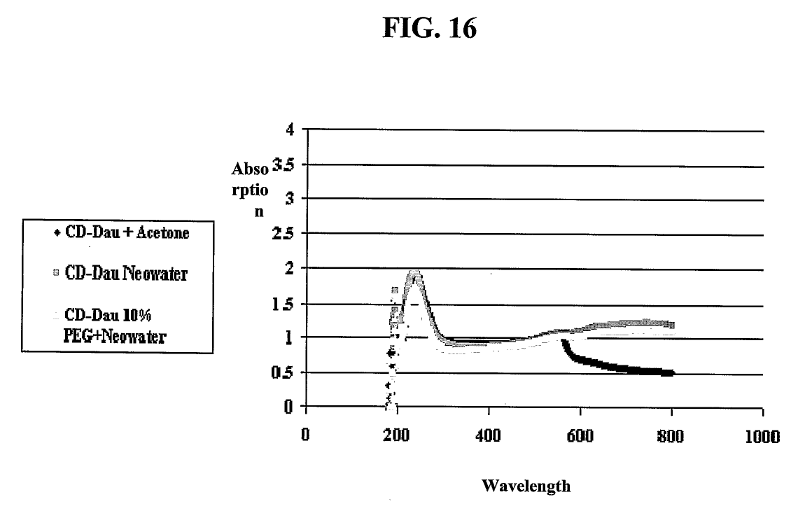

Spectrophotometer measurements (FIG. 16) illustrate the behavior of the material both in the presence and absence of acetone. With acetone there are two peaks in comparison to the material that is suspended with water or with 10% PEG, which in both cases display only one peak.

B. Solubilizing CD-Dau—Part 2:

As soon as the acetone was evaporated from solutions #2, 4 and 5, the material sedimented slightly and an additional amount of acetone was added to the vials. This protocol enables the dissolving of the material in the presence of acetone and Neowater™ while at the same time enabling the subsequent evaporation of acetone from the solution (this procedure was performed twice). Following the second cycle the liquid phase was removed from the vile and additional amount of acetone was added to the sediment material. Once the sediment material dissolved it was merged with the liquid phase removed previously. The merged solution was evaporated again. The solution from vial #1 was removed since the material did not dissolve at all and instead 1.2 ml of acetone was added to the sediment to dissolve the material. Later 1.2 ml of 10% PEG+Neowater™ were added also and after some time the acetone was evaporated from the solution. Finalizing these procedures, the vials were merged to one vial (total volume of 3 ml). On top of this final volume 3 ml of acetone were added in order to dissolve the material and to receive a lucid liquefied solution, which was then evaporated again at 50° C. The solution didn't reach equilibrium due to the fact that once reaching such status the solution would have been separated. By avoiding equilibrium, the material hydration status was maintained and kept as liquid. After the solvent evaporated the material was transferred to a clean vial and was closed under vacuum conditions.

C. Solubilizing CD-Dau—Part 3:

Another 3 ml of the material (total volume of 6 ml) was generated with the addition of 2 ml of acetone-dissolved material and 1 ml of the remaining material left from the previous experiments.

1.9 ml Neowater™ was added to the vial that contained acetone.

100 μl acetone+100 μl Neowater™ were added to the remaining material.

Evaporation was performed on a hot plate adjusted to 50° C.

This procedure was repeated 3 times (addition of acetone and its evaporation) until the solution was stable.

The two vials were merged together.

Following the combining of these two solutions, the materials sedimented slightly. Acetone was added and evaporation of the solvent was repeated.

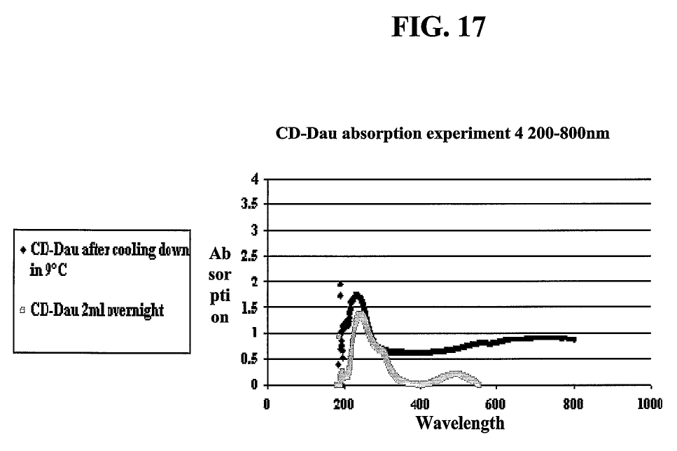

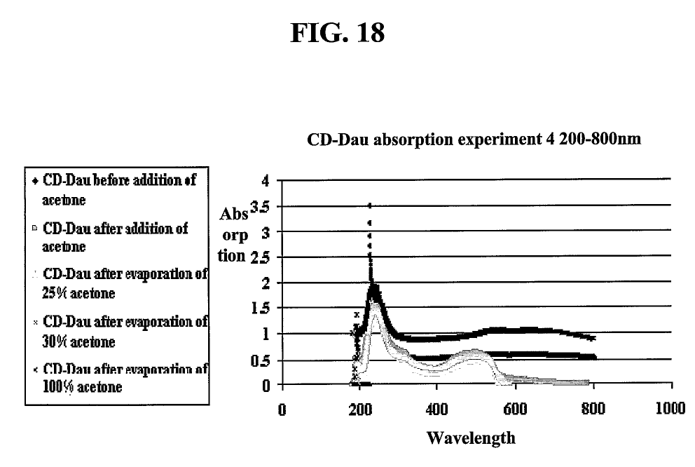

Before mixing the vials (3 ml+2 ml) the first solution prepared in the experiment as described in part 2, hereinabove was incubated at 9° C. over night so as to ensure the solution reached and maintained equilibrium. By doing so, the already dissolved material should not sediment. The following morning the solution's absorption was established and a different graph was obtained (FIG. 17). Following merging of the two vials, absorption measurements were performed again because the material sediment slightly. As a result of the partial sedimentation, the solution was diluted 1:1 by the addition of acetone (5 ml) and subsequently evaporation of the solution was performed at 50° C. on a hot plate. The spectrophotometer read-out of the solution, while performing the evaporation procedure changed due to the presence of acetone (FIG. 18). These experiments imply that when there is a trace of acetone it might affect the absorption readout is received.

B. Solubilizing Daunorubicine (Cerubidine Hydrochloride)

Required concentration: 2 mg/ml

Materials and Methods

2 mg Daunorubicine+1 ml Neowater™ was prepared in one vial and 2 mg of Daunorubicine+1 ml RO was prepared in a second vial.

Results

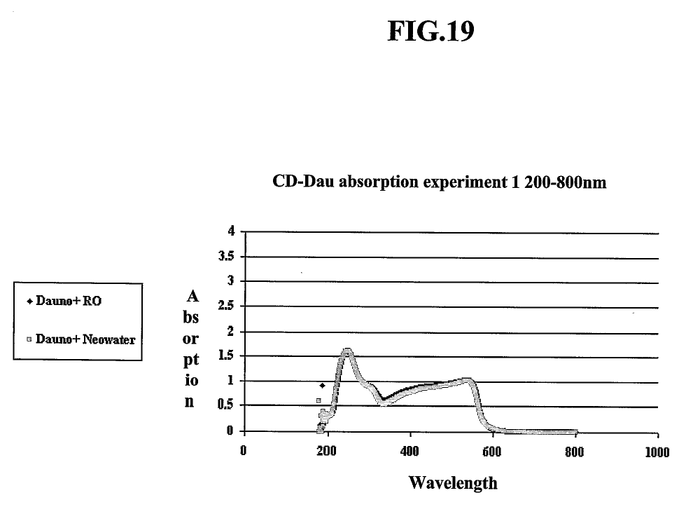

The material dissolved easily both in Neowater™ and RO as illustrated by the spectrophotometer measurements (FIG. 19).

Conclusion

Daunorubicine dissolves without difficulty in Neowater™ and RO.

C. Solubilizing t-boc

Required concentration: 4 mg/ml

Materials and Methods

1.14 ml of EtOH was added to one glass vial containing 18.5 mg of t-boc (an oily material). This was then divided into two vials and 1.74 ml Neowater™ or RO water was added to the vials such that the solution comprised 25% EtOH. Following spectrophotometer measurements, the solvent was evaporated from the solution and Neowater™ was added to both vials to a final volume of 2.31 ml in each vial. The solutions in the two vials were merged to one clean vial and packaged for shipment under vacuum conditions.

Results

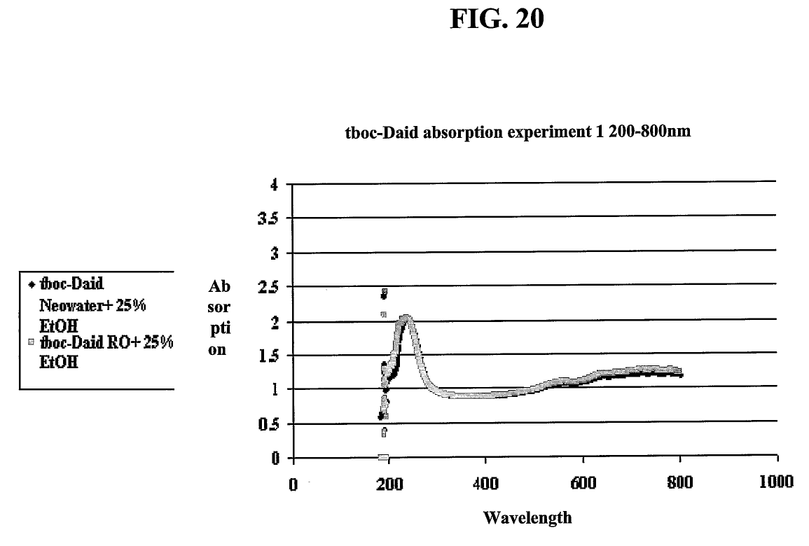

The spectrophotometer measurements are illustrated in FIG. 20. The material dissolved in ethanol. Following addition of Neowater™ and subsequent evaporation of the solvent with heat (50° C.), the material could be dissolved in Neowater™.

Conclusions

The optimal method to dissolve the materials was first to dissolve the material with a solvent (Acetone, Acetic-Acid or Ethanol) followed by the addition of the hydrophilic fluid (Neowater™) and subsequent removal of the solvent by heating the solution and evaporating the solvent.

### Example 10

### Capability of the Liquid Composition Comprising Nanostructures to Dissolve AG-14A and AG-14B

The following experiments were performed in order to ascertain whether the composition comprising nanostructures was capable of dissolving two herbal materials—AG-14A and AG-14B, both of which are known not to dissolve in water at a concentration of 25 mg/ml.

Part 1

Materials and Methods

2.5 mg of each material (AG-14A and AG-14B) was diluted in either Neowater™ alone or a solution comprising 75% Neowater™ and 25% ethanol, such that the final concentration of the powder in each of the four tubes was 2.5 mg/ml. The tubes were vortexed and heated to 50° C. so as to evaporate the ethanol.

Results

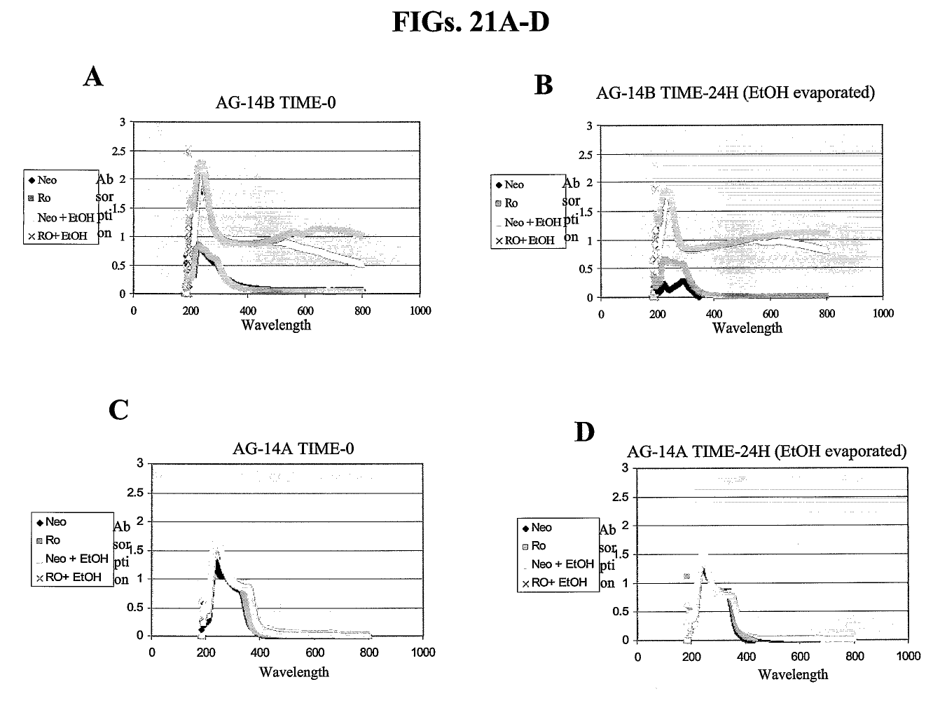

The spectrophotometric measurements of the two herbal materials in Neowater™ in the presence and absence of ethanol are illustrated in FIGS. 21A-D.

Conclusion

Suspension in RO did not dissolve of AG-14B. Suspension of AG-14B in Neowater™ did not aggregate, whereas in RO water, it did.

AG-14A and AG-14B did not dissolve in Neowater/RO.

Part 2

Material and Methods

5 mg of AG-14A and AG-14B were diluted in 62.5 μl EtOH+187.5 μl Neowater™. A further 62.5 μl of Neowater™ were added. The tubes were vortexed and heated to 50° C. so as to evaporate the ethanol.

Results

Suspension in EtOH prior to addition of Neowater™ and then evaporation thereof dissolved AG-14A and AG-14B.

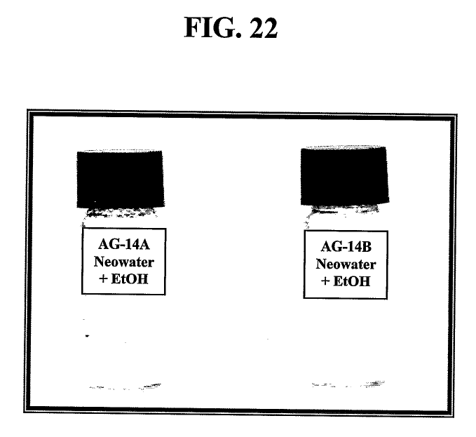

As illustrated in FIG. 22, AG-14A and AG-14B remained stable in suspension for over 48 hours.

### Example 11

### Capability of the Composition Comprising Nanostructures to Dissolve Peptides

The following experiments were performed in order to ascertain whether the composition comprising nanostructures was capable of dissolving 7 cytotoxic peptides, all of which are known not to dissolve in water. In addition, the effect of the peptides on Skov-3 cells was measured in order to ascertain whether the carrier composition comprising nanostructures influenced the cytotoxic activity of the peptides.

Materials and Methods

Solubilization: All seven peptides (Peptide X, X-5FU, NLS-E, Palm-PFPSYK (CMFU), PFPSYKLRPG-NH₂, NLS-p2-LHRH, and F-LH-RH-palm kGFPSK) were dissolved in Neowater™ at 0.5 mM. Spectrophotometric measurements were taken.

In Vitro Experiment: Skov-3 cells were grown in McCoy's 5A medium, and diluted to a concentration of 1500 cells per well, in a 96 well plate. After 24 hours, 2 μl (0.5 mM, 0.05 mM and 0.005 mM) of the peptide solutions were diluted in 1 ml of McCoy's 5A medium, for final concentrations of 10⁻⁶ M, 10⁻⁷ M and 10⁻⁸ M respectively. 9 repeats were made for each treatment. Each plate contained two peptides in three concentration, and 6 wells of control treatment. 90 μl of McCoy's 5A medium+peptides were added to the cells. After 1 hour, 10 μl of FBS were added (in order to prevent competition). Cells were quantified after 24 and 48 hours in a viability assay based on crystal violet. The dye in this assay stains DNA. Upon solubilization, the amount of dye taken up by the monolayer was quantified in a plate reader.

Results

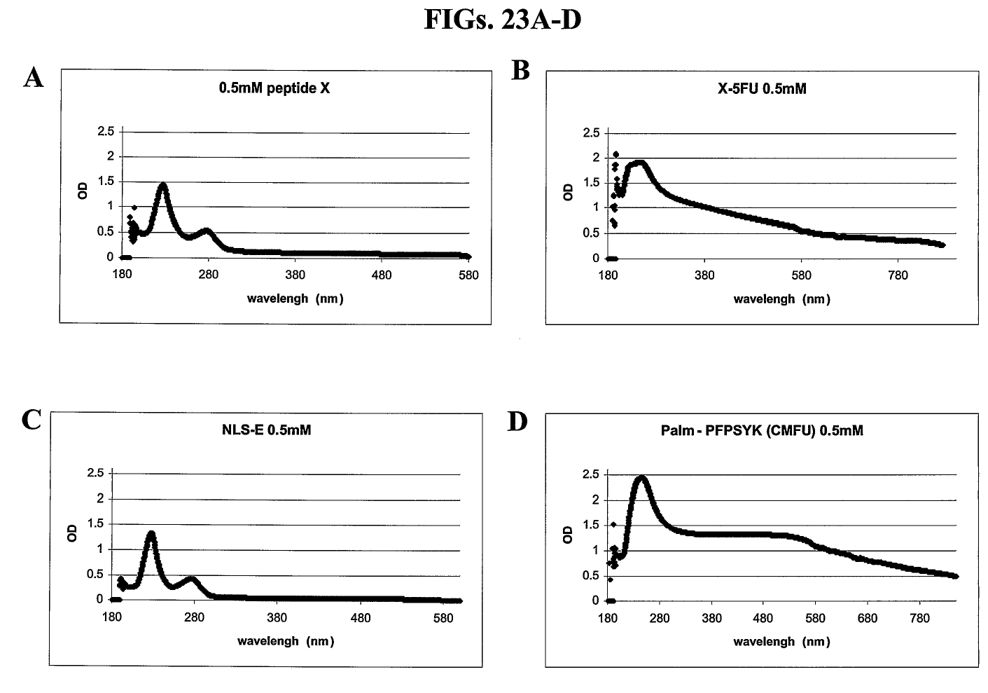

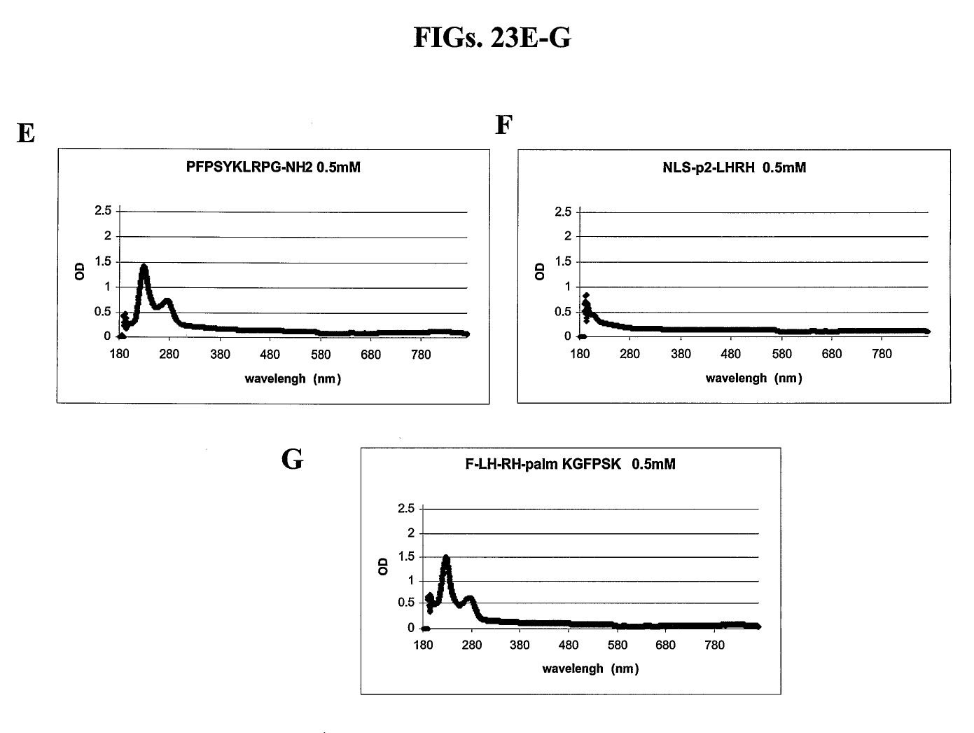

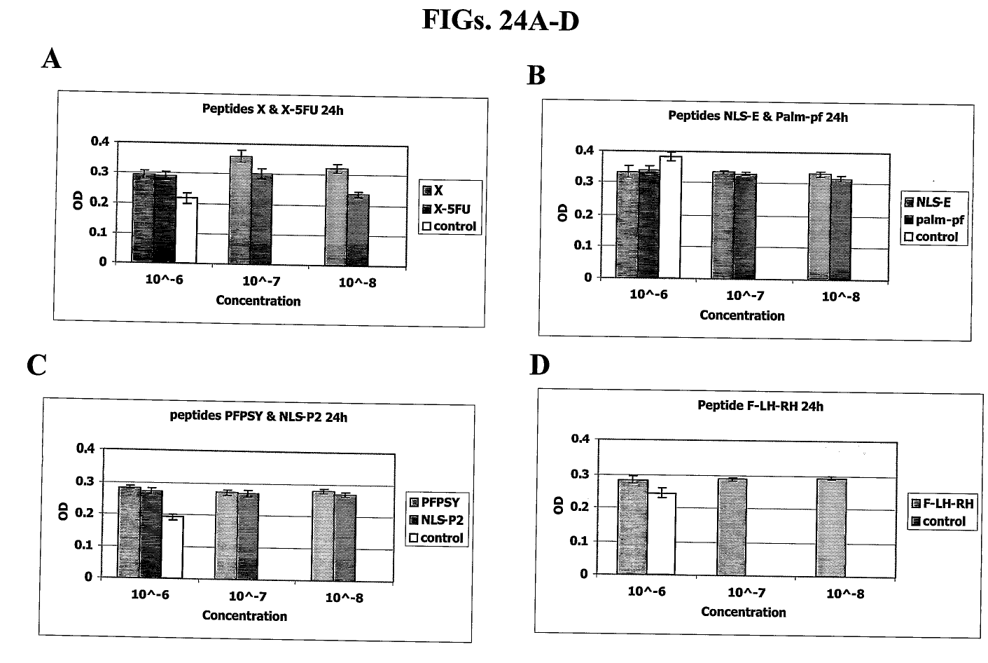

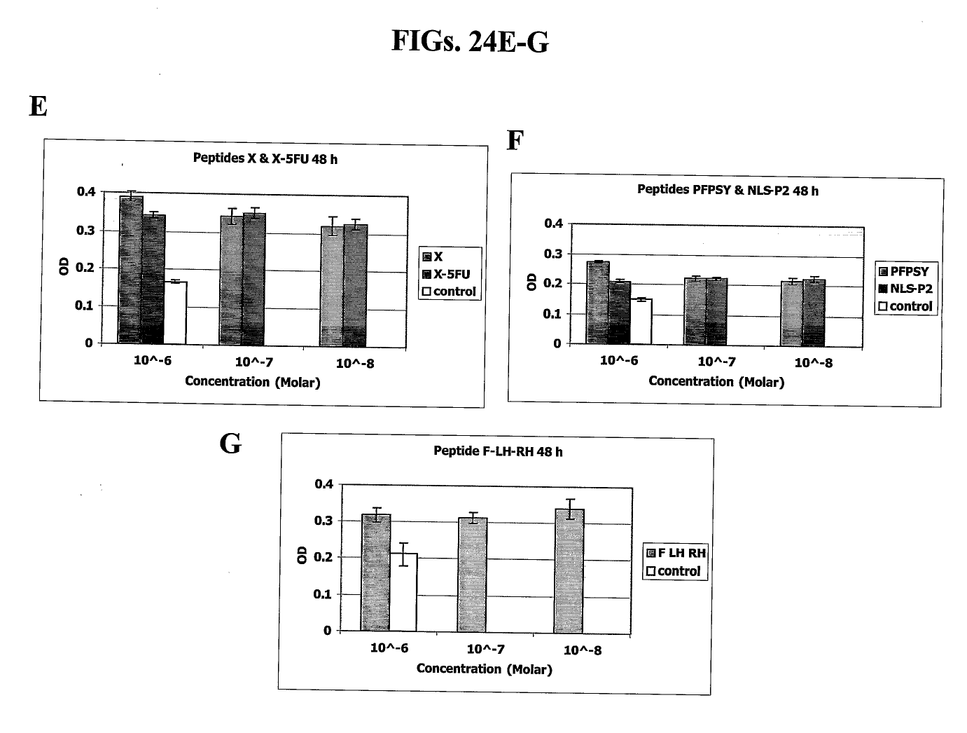

The spectrophotometric measurements of the 7 peptides diluted in Neowater™ are illustrated in FIGS. 23A-G. As illustrated in FIGS. 24A-G, all the dissolved peptides comprised cytotoxic activity.

### Example 12

### Capability of the Liquid Composition Comprising Nanostructures to Dissolve Retinol

The following experiments were performed in order to ascertain whether the liquid composition comprising nanostructures was capable of dissolving retinol.

Materials and Methods

Retinol (vitamin A) was purchased from Sigma (Fluka, 99% HPLC). Retinol was solubilized in Neowater™ under the following conditions.

1% retinol (0.01 gr in 1 ml) in EtOH and Neowater™

0.5% retinol (0.005 gr in 1 ml) in EtOH and Neowater™

0.5% retinol (0.125 gr in 25 ml) in EtOH and Neowater™.

0.25% retinol (0.0625 gr in 25 ml) in EtOH and Neowater™. Final EtOH concentration: 1.5%

Absorbance spectrum of retinol in EtOH: Retinol solutions were made in absolute EtOH, with different retinol concentrations, in order to create a calibration graph; absorbance spectrum was detected in a spectrophotometer.

2 solutions with 0.25% and 0.5% retinol in Neowater™ with unknown concentration of EtOH were detected in a spectrophotometer. Actual concentration of retinol is also unknown since some oil drops are not dissolved in the water.

Filtration: 2 solutions of 0.25% retinol in Neowater™ were prepared, with a final EtOH concentration of 1.5%. The solutions were filtrated in 0.44 and 0.2 μl filter.

Results

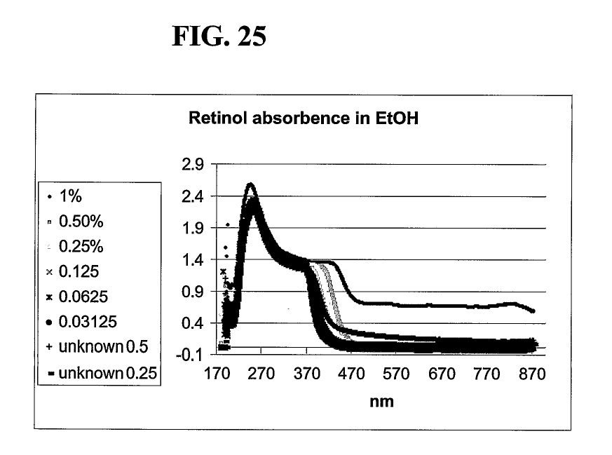

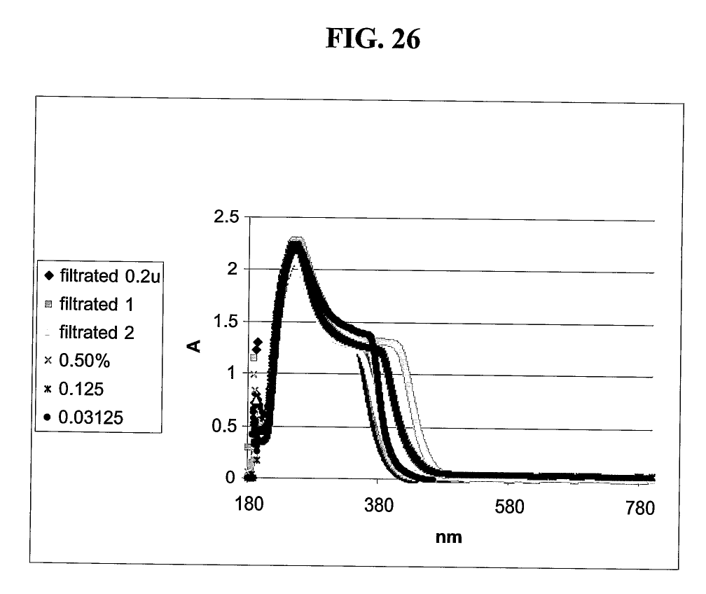

Retinol solubilized easily in alkali Neowater™ rather than acidic Neowater™. The color of the solution was yellow, which faded over time. In the absorbance experiments, 0.5% retinol showed a similar pattern to 0.125% retinol, and 0.25% retinol shows a similar pattern to 0.03125% retinol—see FIG. 25. Since Retinol is unstable in heat; (its melting point is 63° C.), it cannot be autoclaved. Filtration was possible when retinol was fully dissolved (in EtOH). As illustrated in FIG. 26, there is less than 0.03125% retinol in the solutions following filtration. Both filters gave similar results.

### Example 13

### Capability of the Liquid Composition Comprising Nanostructures to Dissolve Material X

The following experiments were performed in order to ascertain whether the liquid composition comprising nanostructures was capable of dissolving material X at a final concentration of 40 mg/ml.

Part 1—Solubility in Water and DMSO

Materials and Methods

In a first test tube, 25 μl of Neowater™ was added to 1 mg of material “X”. In a second test tube 25 μl of DMSO was added to 1 mg of material “X”. Both test tubes were vortexed and heated to 60° C. and shaken for 1 hour on a shaker.

Results

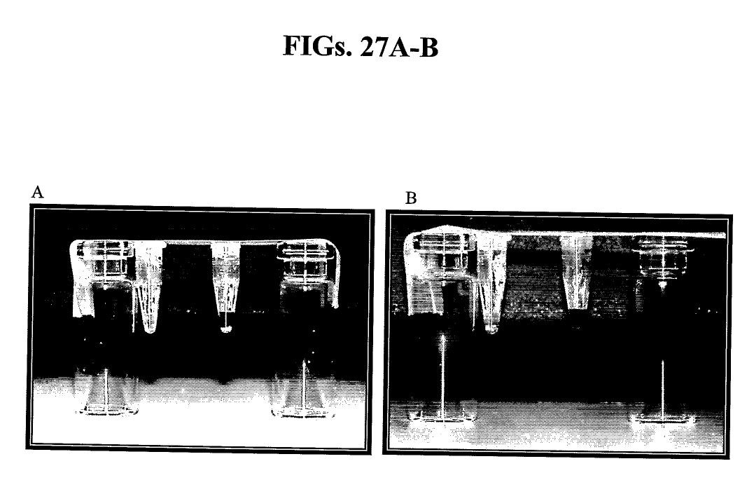

The material did not dissolve at all in Neowater™ (test tube 1). The material dissolved in DMSO and gave a brown-yellow color. The solutions remained for 24-48 hours and their stability was analyzed over time (FIG. 27A-B).

Conclusions

Neowater™ did not dissolve material “X” and the material sedimented, whereas DMSO almost completely dissolved material “X”.

Part 2—Reduction of DMSO and Examination of the Material Stability/Kinetics in Different Solvents Over the Course of Time.

Materials and Methods

6 different test tubes were analyzed each containing a total reaction volume of 25 μl:

1. 1 mg “X”+25 μl Neowater™ (100%).

2. 1 mg “X”+12.5 μl DMSO→12.5 μl Neowater™ (50%).

3. 1 mg “X”+12.5 μl DMSO+12.5 μl Neowater™ (50%).

4. 1 mg “X”+6.25 μl DMSO+18.75 μl Neowater™ (25%).

5. 1 mg “X”+25 μl Neowater™+sucrose* (10%).

6. 1 mg+12.5 μl DMSO+12.5 μl dehydrated Neowater™ ** (50%).

* 0.1 g sucrose+1 ml (Neowater™)=10% Neowater™+sucrose
** Dehydrated Neowater™ was achieved by dehydration of Neowater™ for 90 min at 60° C.

All test tubes were vortexed, heated to 60° C. and shaken for 1 hour.

Results

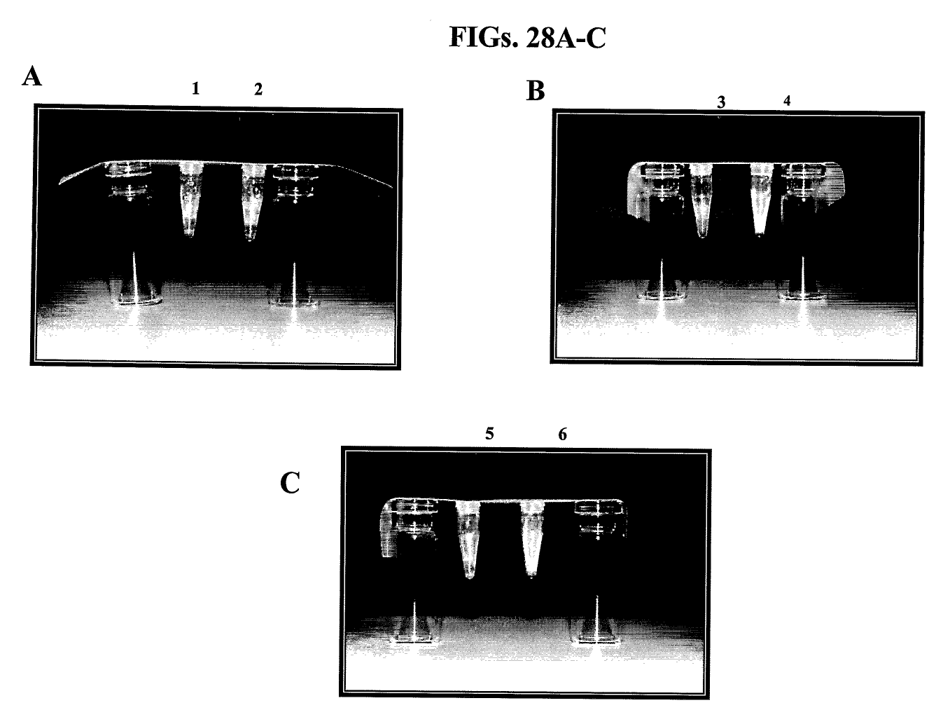

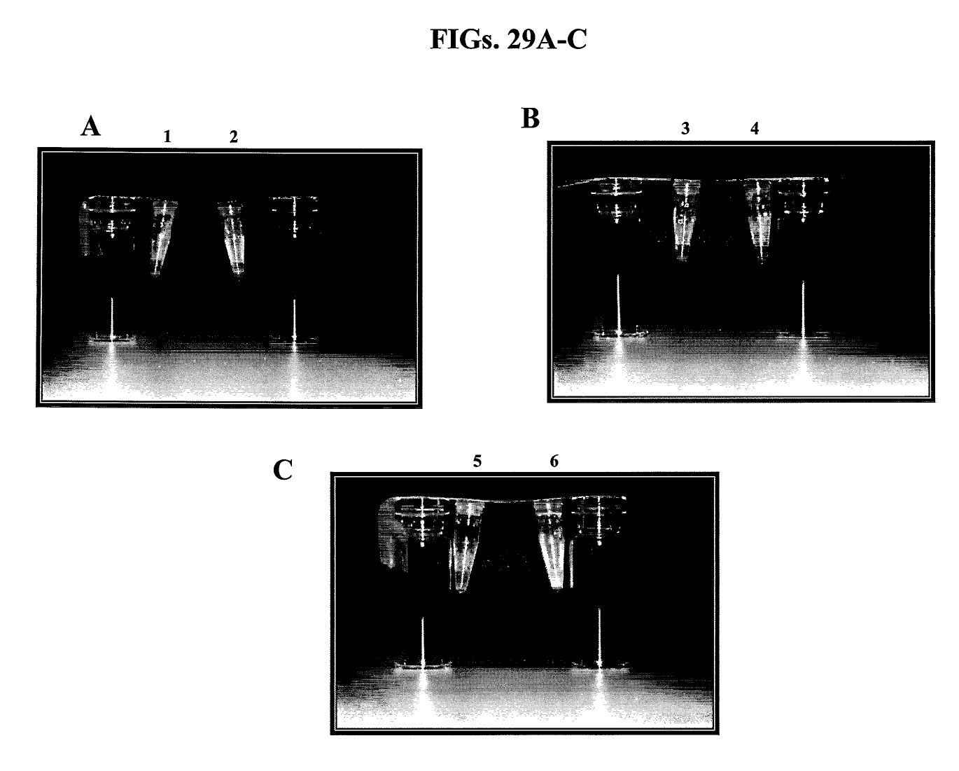

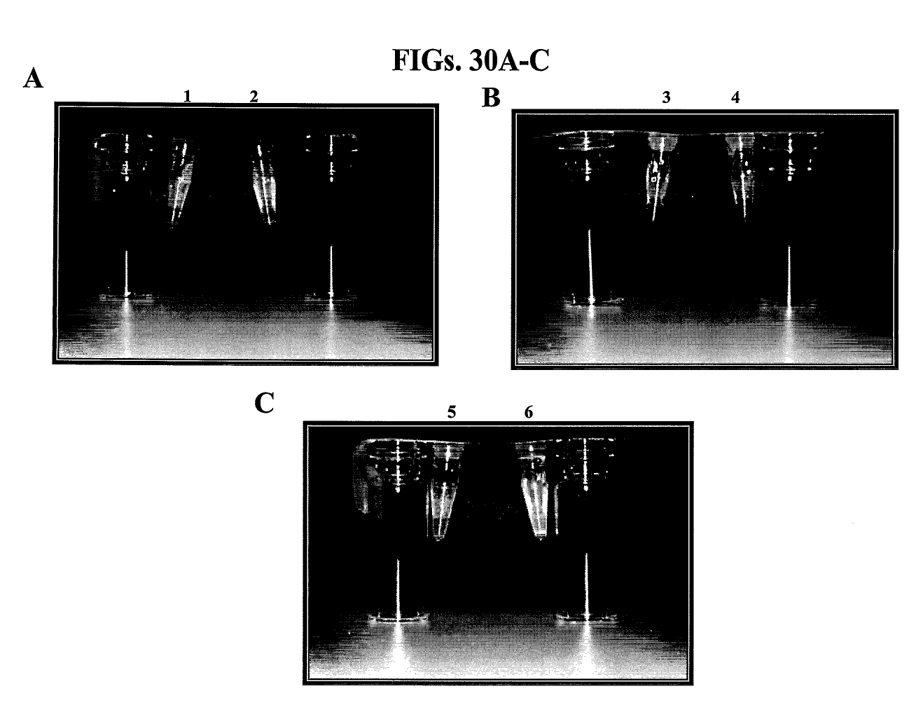

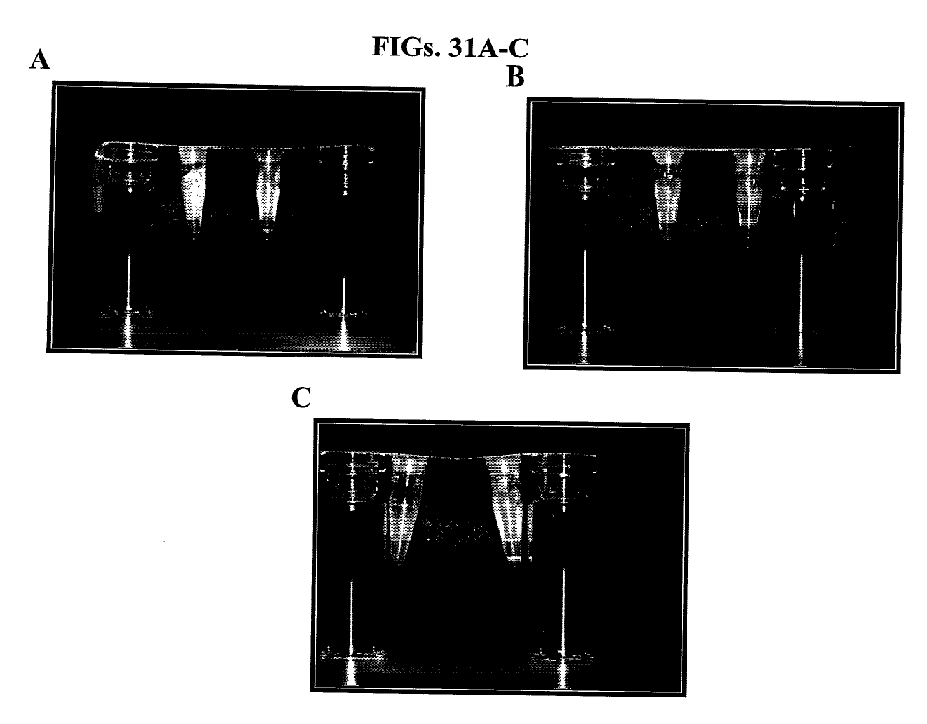

The test tubes comprising the 6 solvents and substance X at time 0 are illustrated in FIGS. 28A-C. The test tubes comprising the 6 solvents and substance X at 60 minutes following solubilization are illustrated in FIGS. 29A-C. The test tubes comprising the 6 solvents and substance X at 120 minutes following solubilization are illustrated in FIGS. 30A-C. The test tubes comprising the 6 solvents and substance X 24 hours following solubilization are illustrated in FIGS. 31A-C.

Conclusion

Material “X” did not remain stable throughout the course of time, since in all the test tubes the material sedimented after 24 hours.

There is a different between the solvent of test tube 2 and test tube 6 even though it contains the same percent of solvents. This is because test tube 6 contains dehydrated Neowater™ which is more hydrophobic than non-dehydrated Neowater™.

Part 3 Further Reduction of DMSO and Examination of the Material Stability/Kinetics in Different Solvents Over the Course of Time.

Materials and Methods

1 mg of material “X”+50 μl DMSO were placed in a glass tube. 50 μl of Neowater™ were titred (every few seconds 5 μl) into the tube, and then 500 μl of a solution of Neowater™ (9% DMSO+91% Neowater™) was added.

In a second glass tube, 1 mg of material “X”+50 μl DMSO were added. 50 μl of RO were titred (every few seconds 5 μl) into the tube, and then 500 μl of a solution of RO (9% DMSO+91% RO) was added.

Results

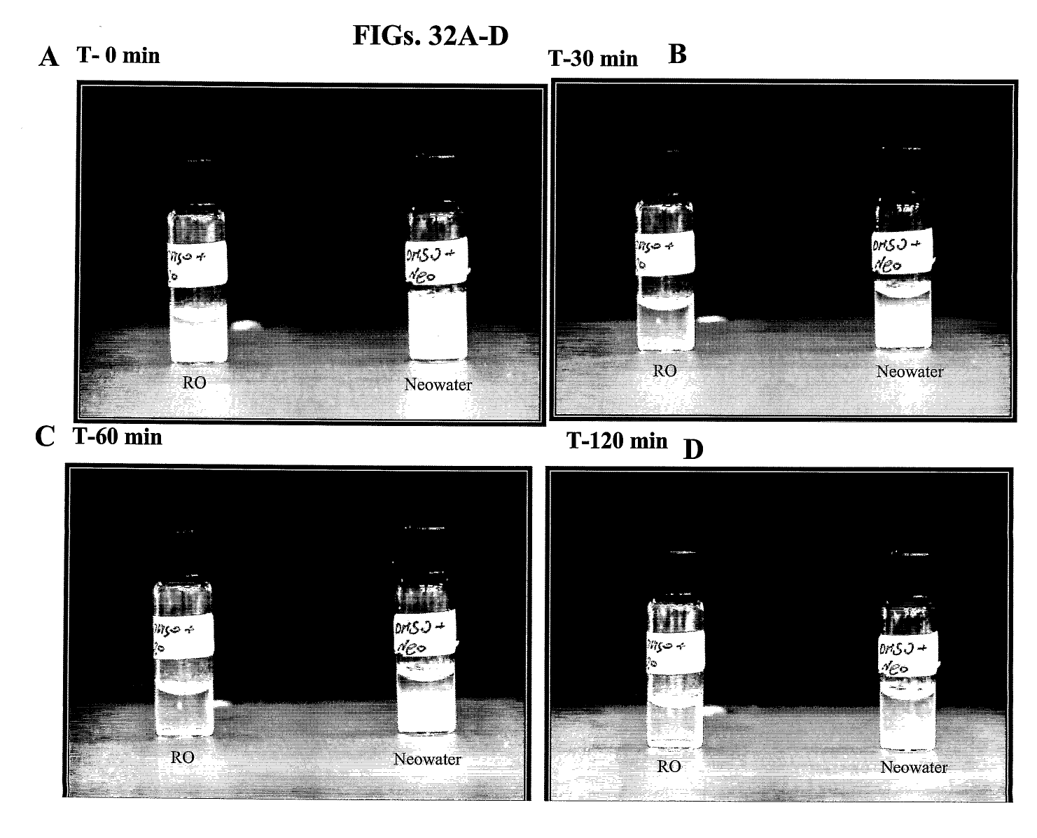

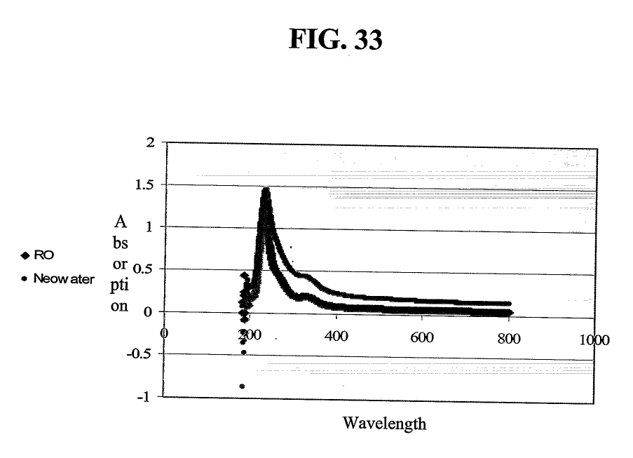

As illustrated in FIGS. 32A-D, material “X” remained dispersed in the solution comprising Neowater™, but sedimented to the bottom of the tube, in the solution comprising RO water. FIG. 33 illustrates the absorption characteristics of the material dispersed in RO/Neowater™ and acetone 6 hours following vortexing.

Conclusion

It is clear that material “X” dissolves differently in RO compare to Neowater™, and it is more stable in Neowater™ compare to RO. From the spectrophotometer measurements (FIG. 33), it is apparent that the material “X” dissolved better in Neowater™ even after 5 hours, since, the area under the graph is larger than in RO. It is clear the Neowater™ hydrates material “X”. The amount of DMSO may be decreased by 20-80% and a solution based on Neowater™ may be achieved that hydrates material “X” and disperses it in the Neowater™.

### Example 14

### Capability of the Liquid Composition Comprising Nanostructures to Dissolve SPL 2101 and SPL 5217

The following experiments were performed in order to ascertain whether the liquid composition comprising nanostructures was capable of dissolving material SPL 2101 and SPL 5217 at a final concentration of 30 mg/ml.

Materials and Methods

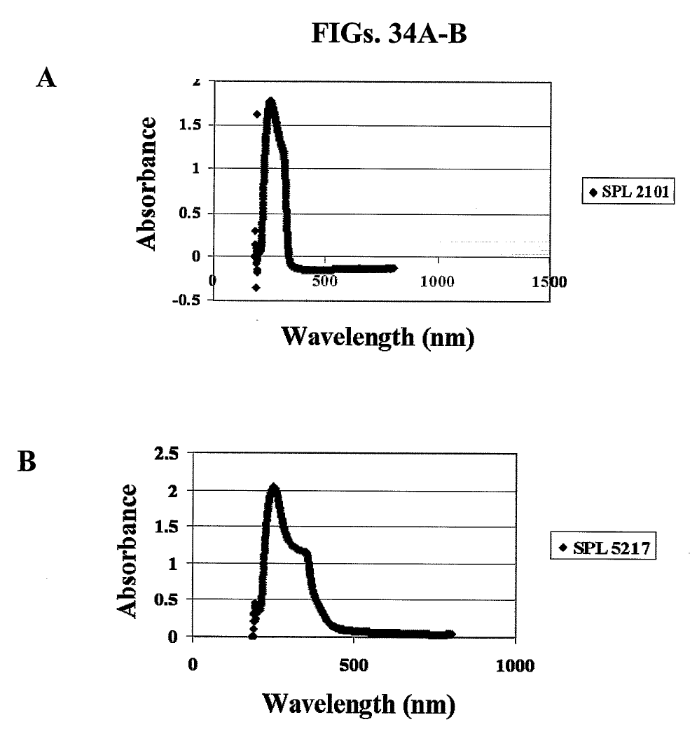

SPL 2101 was dissolved in its optimal solvent (ethanol)—FIG. 34A and SPL 5217 was dissolved in its optimal solvent (acetone)—FIG. 34B. The two compounds were put in glass vials and kept in dark and cool environment. Evaporation of the solvent was performed in a dessicator and over a long period of time Neowater™ was added to the solution until there was no trace of the solvents.

Results

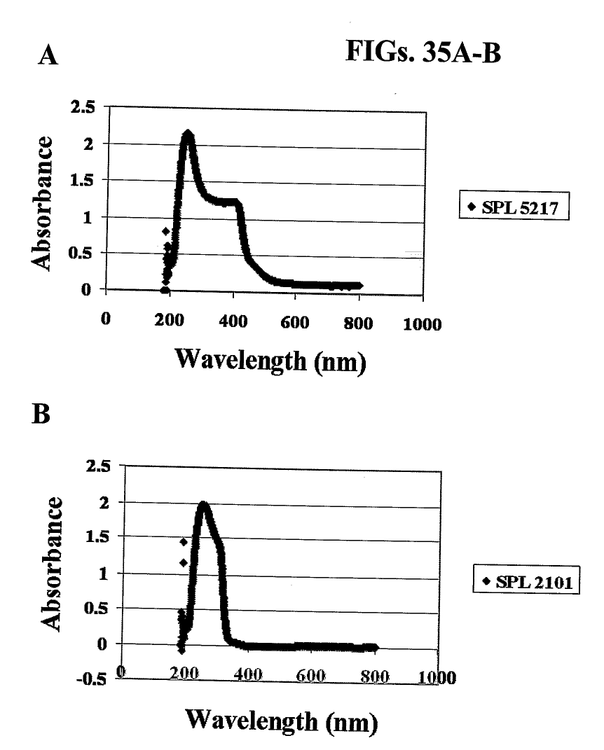

SPL 2101 & SPL 5217 dissolved in Neowater™ M as illustrated by the spectrophotometer data in FIGS. 35A-B.

### Example 15

### Capability of the Liquid Composition Comprising Nanostructures to Dissolve Taxol

The following experiments were performed in order to ascertain whether the composition comprising nanostructures was capable of dissolving material taxol (Paclitaxel) at a final concentration of 0.5 mM.

Materials and Methods

Solubilization: 0.5 mM Taxol solution was prepared (0.0017 gr in 4 ml) in either DMSO or Neowater™ with 17% EtOH. Absorbance was detected with a spectrophotometer.

Cell viability assay: 150,000 293T cells were seeded in a 6 well plate with 3 ml of DMEM medium. Each treatment was grown in DMEM medium based on RO or Neowater™. Taxol (dissolved in Neowater™ or DMSO) was added to final concentration of 1.666 μM (10 μl of 0.5 mM Taxol in 3 ml medium). The cells were harvested following a 24 hour treatment with taxol and counted using tryptan blue solution to detect dead cells.

Results

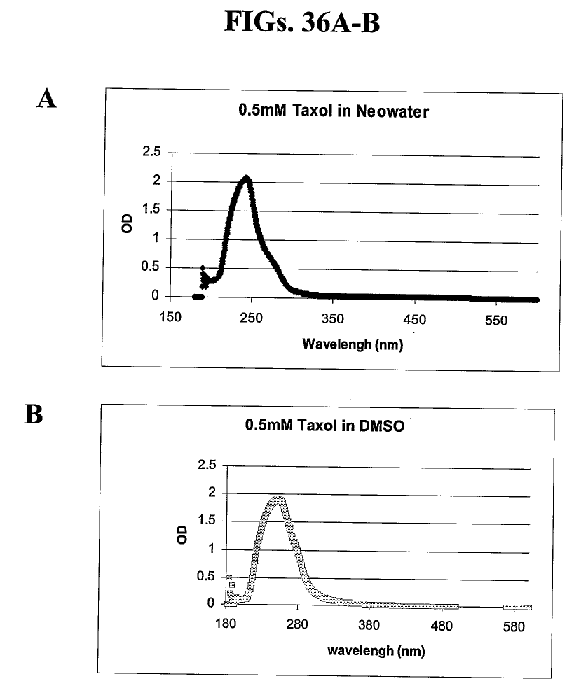

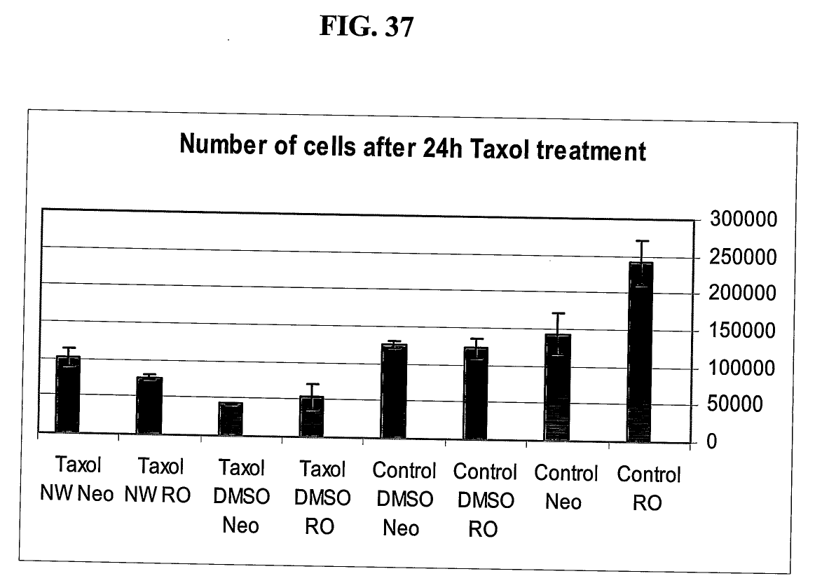

Taxol dissolved both in DMSO and Neowater™ as illustrated in FIGS. 36A-B. The viability of the 293T cells following various solutions of taxol is illustrated in FIG. 37.

Conclusion

Taxol comprised a cytotoxic effect following solution in Neowater™.

### Example 16

### Stabilizing Effect of the Liquid Composition Comprising Nanostructures

The following experiment was performed to ascertain if the liquid composition comprising nanostructures effected the stability of a protein.

Materials and Methods

Two commercial Taq polymerase enzymes (Peq-lab and Bio-lab) were checked in a PCR reaction to determine their activities in ddH₂O (RO) and carrier comprising nanostructures (Neowater™—Do-Coop technologies, Israel). The enzyme was heated to 95° C. for different periods of time, from one hour to 2.5 hours. 2 types of reactions were made:

Water only—only the enzyme and water were boiled.

All inside—all the reaction components were boiled: enzyme, water, buffer, dNTPs, genomic DNA and primers.

Following boiling, any additional reaction component that was required was added to PCR tubes and an ordinary PCR program was set with 30 cycles.

Results

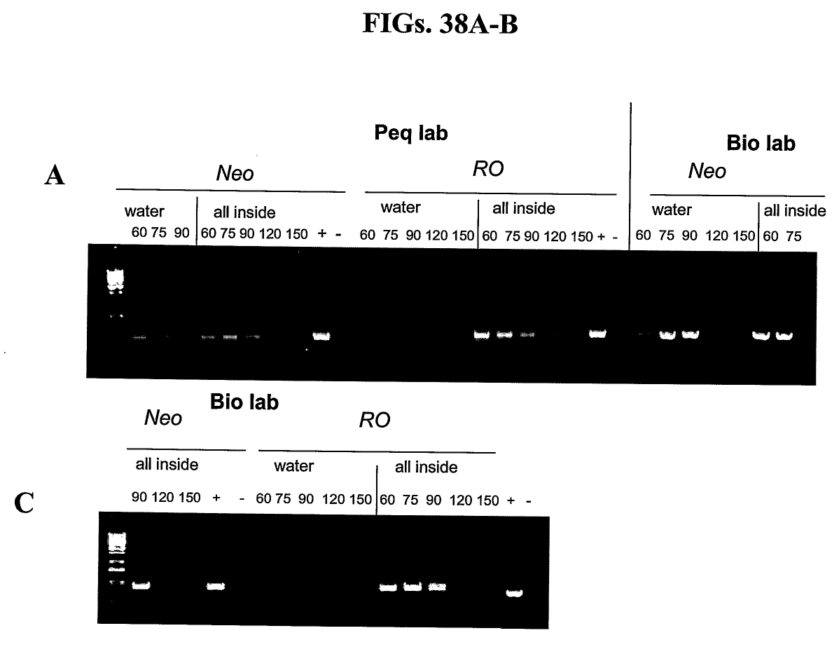

As illustrated in FIGS. 38A-B, the carrier composition comprising nanostructures protected the enzyme from heating, both under conditions where all the components were subjected to heat stress and where only the enzyme was subjected to heat stress. In contrast, RO water only protected the enzyme from heating under conditions where all the components were subjected to heat stress.

### Example 17

### Further Illustration of the Stabilizing Effect of the Carrier Comprising Nanostructures

The following experiment was performed to ascertain if the carrier composition comprising nanostructures effected the stability of two commercial Taq polymerase enzymes (Peq-lab and Bio-lab).

Materials and Methods

The PCR reactions were set up as follows:

Peq-lab samples: 20.4 μl of either the carrier composition comprising nanostructures (Neowater™—Do-Coop technologies, Israel) or distilled water (Reverse Osmosis=RO).

0.1 μl Taq polymerase (Peq-lab, Taq DNA polymerase, 5 U/μl)

Three samples were set up and placed in a PCR machine at a constant temperature of 95° C. Incubation time was: 60, 75 and 90 minutes.

Following boiling of the Taq enzyme the following components were added:

2.5 μl 10× reaction buffer Y (Peq-lab)
0.5 μl dNTPs 10 mM (Bio-lab)
1 μl primer GAPDH mix 10 pmol/μl
0.5 μl genomic DNA 35 μg/μl

Biolab Samples

18.9 μl of either carrier comprising nanostructures (Neowater™—Do-Coop technologies, Israel) or distilled water (Reverse Osmosis=RO).

0.1 μl Taq polymerase (Bio-lab, Taq polymerase, 5 U/μl)

Five samples were set up and placed in a PCR machine at a constant temperature of 95° C. Incubation time was: 60, 75, 90 120 and 150 minutes.

Following boiling of the Taq enzyme the following components were added:

2.5 μl TAQ 10× buffer Mg-free (Bio-lab)

### 1.5 μl MgCl₂ 25 mM (Bio-lab)

0.5 μl dNTPs 10 mM (Bio-lab)
1 μl primer GAPDH mix (10 pmol/μl)
0.5 μl genomic DNA (35 μg/μl)

For each treatment (Neowater or RO) a positive and negative control were made. Positive control was without boiling the enzyme. Negative control was without boiling the enzyme and without DNA in the reaction. A PCR mix was made for the boiled taq assays as well for the control reactions.

Samples were placed in a PCR machine, and run as follows:

PCR Program:

1. 94° C. 2 minutes denaturation

2. 94° C. 30 seconds denaturation

3. 60° C. 30 seconds annealing

4. 72° C. 30 seconds elongation

repeat steps 2-4 for 30 times

5. 72° C. 10 minutes elongation

Results

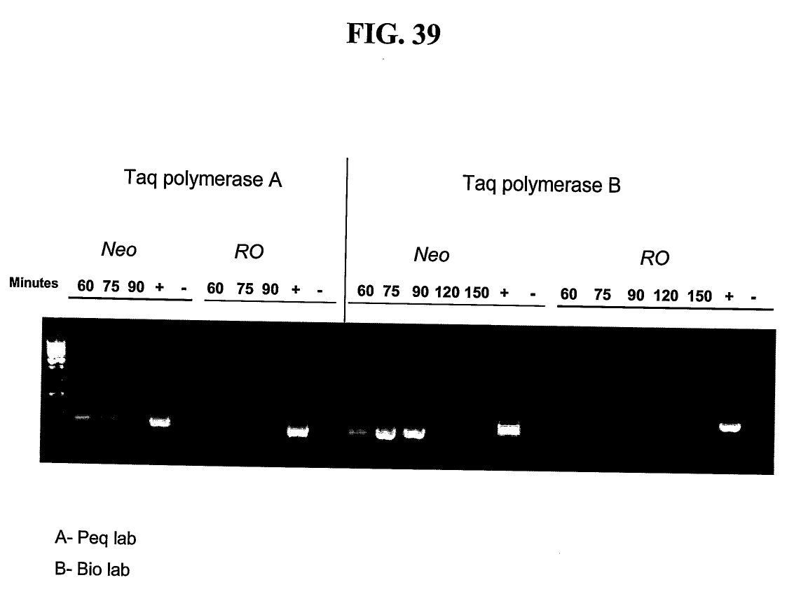

As illustrated in FIG. 39, the liquid composition comprising nanostructures protected both the enzymes from heat stress for up to 1.5 hours.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An antiseptic composition comprising at least one antiseptic agent and a carrier composition comprising nanostructures and a liquid.

2. A method of disinfecting a body surface of an individual comprising providing to an individual in need thereof an antiseptic effective amount of a composition wherein said composition comprises nanostructures and a liquid, thereby disinfecting a body surface of an individual.

3. A method of sterilizing an object comprising contacting the object with a composition comprising nanostructures and a liquid, thereby sterilizing the object.

4. The method of claim 2, wherein the composition further comprises at least one antiseptic agent.

5-17. (canceled)

18. The antiseptic composition claim 1, wherein said nanostructures are formulated from hydroxyapatite.

19. The antiseptic composition of claim 1, being formulated as a liquid composition.

20. (canceled)

21. The antiseptic composition of claim 1, being formulated as a solid composition.

22. (canceled)

23. The antiseptic composition of claim 1, being formulated as a semi-solid composition.

24. (canceled)

25. The antiseptic composition of claim 1, being formulated as an oral dosage form.

26. (canceled)

27. The antiseptic composition of claim 1, being formulated as a topical or mucosal dosage form.

28. (canceled)

29. The antiseptic composition of claim 1, comprising less than 20% by volume alcohol.

30. The antiseptic composition of claim 1 being devoid of alcohol.

31. The antiseptic composition of claim 1, wherein said at least one antiseptic agent is an orally non-toxic antiseptic agent.

32. (canceled)

33. The antiseptic composition of claim 1, wherein said at least one antiseptic agent is selected from the group consisting of a monohydric alcohol, a metal compound, a quaternary ammonium compound, iodine, an iodophor and a phenolic compound.

34-37. (canceled)

38. The method of claim 2, wherein said body surface is a skin, a tooth or a mucous membrane.

39. The method of claim 3, wherein said antiseptic agent is a toxic agent.

40. (canceled)

41. The method of claim 3, wherein the composition further comprises at least one antiseptic agent.

42. The method of claim 2, wherein said at least one antiseptic agent is an orally non-toxic antiseptic agent.

43. The method of claim 2, wherein said at least one antiseptic agent is selected from the group consisting of a monohydric alcohol, a metal compound, a quaternary ammonium compound, iodine, an iodophor and a phenolic compound.

44. The method of claim 3, wherein said at least one antiseptic agent is selected from the group consisting of a monohydric alcohol, a metal compound, a quaternary ammonium compound, iodine, an iodophor and a phenolic compound.
